(19) United States
(12) Patent Application Publication
(10) Pub. No.: US 2022/0267272 A1
(43) Pub. Date: Aug. 25, 2022

(54) PYRIDINIUM SALT AND PEST CONTROL AGENT

(71) Applicant: Nippon Soda Co., Ltd., Chiyoda-ku, Tokyo (JP)

(72) Inventors: Hiroto SUZUKI, Odawara-shi (JP); Riho TAGUCHI, Odawara-shi (JP); Kotaro SHIBAYAMA, Odawara-shi (JP); Takao IWASA, Odawara-shi (JP); Miho TAKAHASHI, Makinohara-shi (JP); Tsuyoshi HOYA, Makinohara-shi (JP); Kento IWATA, Odawara-shi (JP)

(73) Assignee: Nippon Soda Co., Ltd., Chiyoda-ku, Tokyo (JP)

(21) Appl. No.: 17/612,302

(22) PCT Filed: May 26, 2020
(86) PCT No.: PCT/JP2020/020719

§ 371 Date: Nov. 18, 2021

(30) Foreign Application Priority Data
May 29, 2019 (JP) 2019-100657

(51) Int. Cl. C07D 213/89; A01N 43/40

(52) U.S. Cl. CPC C07D 213/89; A01N 43/40

(57) ABSTRACT

Provided are: a compound represented by Formula (I) or Formula (II) (wherein A represents an oxygen atom or a sulfur atom, X¹ represents a substituted or unsubstituted C1-6 alkyl group or the like, m is any integer of 0 to 5, X² represents a C2-8 haloalkyl group or the like, X³ represents a halogeno group or the like, n is any integer of 0-4, Z^{q-} is a counter ion, and q is 1 or 2); and a pest control agent containing the compound as an active agent.

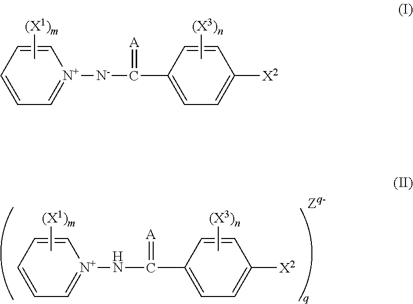

### TECHNICAL FIELD

The present invention relates to a pyridinium salt and a pest control agent. In more detail, the present invention relates to a pyridinium salt that has excellent insecticidal activity and/or acaricidal activity, and excellent safety, and that can be synthesized industrially advantageously, as well as a pest control agent containing the same as an active ingredient thereof.

The present invention claims priority on the basis of Japanese Patent Application No. 2019-100657 filed in Japan on May 29, 2019, the contents of which are incorporated herein by reference.

### BACKGROUND OF THE INVENTION

Various compounds having insecticidal and/or acaricidal activity have been proposed. In the case where such compounds are used as agrochemicals, the compounds are required not only to exhibit high activity, but also to exhibit less possibility of causing chemical resistance, harmful effects on plants, or soil pollution, and to exhibit low toxicity to domestic animals or fish.

Patent Document 1 discloses a compound of formula (A) which has acaricidal activity.

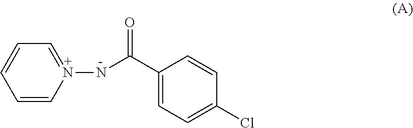

Non-patent Document 1 discloses a compound of formula (B) which has analgesic activity.

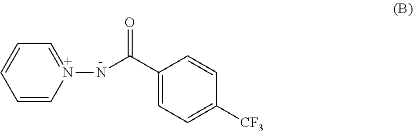

### DOCUMENTS OF RELATED ART

### Patent Documents

Patent Document 1: U.S. Pat. No. 4,138,548

### Non-Patent Documents

Non-patent Document 1: Journal of Medicinal Chemistry, 1982, vol. 25, p 720-72

### SUMMARY OF THE INVENTION

### Problems to be Solved by the Invention

The present invention aims to provide a pyridinium salt that exhibits excellent pest control activity, particularly, insecticidal activity and/or acaricidal activity, and excellent safety, and that can be synthesized industrially advantageously. In addition, the present invention aims to provide a pest control agent, insecticide or acaricide, ectoparasite control agent, endoparasite control agent, or expellant, containing the pyridinium salt as an active ingredient thereof.

### Means to Solve the Problems

The present invention encompassing the following aspects has been completed as a result of studying to solve the above-described problems.

(1) A compound of formula (I) or formula (II).

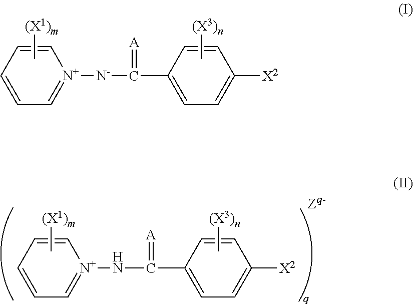

In the formulae (I) and (II),

A is an oxygen atom or a sulfur atom,

X¹ is a halogeno group, a substituted or unsubstituted C1-6 alkyl group, a substituted or unsubstituted C2-6 alkenyl group, a substituted or unsubstituted C2-6 alkynyl group, a substituted or unsubstituted C1-6 alkoxy group, a substituted or unsubstituted C2-6 alkenyloxy group, a substituted or unsubstituted C2-6 alkynyloxy group, a substituted or unsubstituted C1-6 alkylcarbonyl group, a substituted or unsubstituted C1-6 alkoxycarbonyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryl group, a substituted or unsubstituted C6-10 aryloxy group, a substituted or unsubstituted 5- or 6-membered heteroaryl group, a substituted or unsubstituted 5- or 6-membered heteroaryloxy group, a substituted or unsubstituted C6-10 arylcarbonyl group, a substituted or unsubstituted 5- or 6-membered heteroarylcarbonyl group, a group of R^{a}R^{b}N—, a group of R^{a}R^{b}N—CO—, a group of R^{c}CO—NH—, a pentafluorosulfanyl group, a nitro group, or a cyano group,

m indicates the number of X¹ and is an integer of 0 to 5,

R^{a} is a hydrogen atom, a substituted or unsubstituted C1-6 alkyl group, or a substituted or unsubstituted C6-10 aryl group,

R^{b} is a hydrogen atom, a substituted or unsubstituted C1-6 alkyl group, or a substituted or unsubstituted C6-10 aryl group,

R^{c} is a hydrogen atom, a substituted or unsubstituted C1-6 alkyl group, or a substituted or unsubstituted C6-10 aryl group,

X² is a C5-8 alkyl group, a C2-8 haloalkyl group, a (substituted or unsubstituted C6-10 aryl) C1-6 alkyl group, a (substituted or unsubstituted 5- or 6-membered heteroaryl) C1-6 alkyl group, a (substituted or unsubstituted C6-10 aryloxy) C1-6 alkyl group, a (substituted or unsubstituted 5- or 6-membered heteroaryloxy) C1-6 alkyl group, a C3-6 alkenyl group, a C2-6 haloalkenyl group, a (substituted or unsubstituted C6-10 aryl) C2-6 alkenyl group, a (substituted or unsubstituted 5- or 6-membered heteroaryl) C2-6 alkenyl group, a C2-6 alkynyl group, a C2-6 haloalkynyl group, a (substituted or unsubstituted C6-10 aryl) C2-6 alkynyl group, a (substituted or unsubstituted 5- or 6-membered heteroaryl) C2-6 alkynyl group, a hydroxy group, a substituted C1-8 alkoxy group, a substituted or unsubstituted C2-6 alkenyloxy group, a substituted or unsubstituted C2-6 alkynyloxy group, a C2-6 alkylcarbonyl group, a substituted C1-6 alkylcarbonyl group, a substituted or unsubstituted C1-6 alkoxycarbonyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryloxy group, a substituted or unsubstituted 5- or 6-membered heteroaryloxy group, a substituted or unsubstituted C6-arylcarbonyl group, a substituted or unsubstituted 5- or 6-membered heteroarylcarbonyl group, a carbamoyl group, a group of R^{a1}R^{b1}N—, a group of R^{a1}R^{b1}N—CO—, a group of R^{a1}R^{b1}N—CO—N(R^{d1})—, a group of R^{a1}R^{b1}N—CS—N(R^{d1})—, a group of R^{c1}CO—N(R^{d1})—, a group of R^{c1}O—N═CR^{f1}—, a group of R^{g1}SO₂—N(R^{d1})—, a group of R^{g1}SO₂NH—N═CR^{f1}—, or a pentafluorosulfanyl group,

R^{a1} is a substituted or unsubstituted C1-6 alkyl group, or a substituted or unsubstituted C6-10 aryl group,

R^{b1} is a hydrogen atom, a substituted or unsubstituted C1-6 alkyl group, or a substituted or unsubstituted C6-10 aryl group,

R^{a1} and R^{b1} may be bonded together to form a divalent organic group,

R^{c1} is a hydrogen atom, a substituted or unsubstituted C1-6 alkyl group, a substituted or unsubstituted C1-6 alkoxy group, or a substituted or unsubstituted C6-10 aryl group,

R^{d1} is a hydrogen atom, a substituted or unsubstituted C1-6 alkyl group or a substituted or unsubstituted C6-10 aryl group,

R^{e1} is a hydrogen atom, a substituted or unsubstituted C1-6 alkyl group or a substituted or unsubstituted C6-10 aryl group,

R^{f1} is a hydrogen atom or a substituted or unsubstituted C1-6 alkyl group,

R^{g1} is a substituted or unsubstituted C1-6 alkyl group or a substituted or unsubstituted C6-10 aryl group,

X³ is a halogeno group, a substituted or unsubstituted C1-6 alkyl group, a hydroxy group, a substituted or unsubstituted C1-6 alkoxy group, a substituted or unsubstituted C1-6 alkylthio group, a substituted or unsubstituted C1-6 alkylsulfinyl group, a substituted or unsubstituted C1-6 alkylsulfonyl group, a pentafluorosulfanyl group, a nitro group, or a cyano group,

n indicates the number of X³ and is an integer of 0 to 4,

Z^{q-} is a counter ion, and

q indicates the valence of the counter ion and is 1 or 2.

(2) A pest control agent containing at least one selected from the compounds of (1) as an active ingredient thereof.

(3) An insecticide or acaricide containing at least one selected from the compounds of (1) as an active ingredient thereof.

(4) An ectoparasite control agent containing at least one selected from the compounds of (1) as an active ingredient thereof.

(5) An endoparasite control agent or expellant containing at least one selected from the compounds of (1) as an active ingredient thereof.

### Effects of the Invention

The pyridinium salt according to the present invention has control activity against pests that cause problems to agricultural crops or in the health field. The control agent containing the pyridinium salt according to the present invention can effectively control pests, particularly agricultural insect pests and acarians, at low doses, and can further effectively control ectoparasites and endoparasites that may harm humans and animals.

### EMBODIMENTS FOR CARRYING OUT THE INVENTION

The pyridinium salt according to the present invention is a compound of formula (I) (intramolecular salt) or a compound of formula (II) (intermolecular salt). The intramolecular salt is a compound having a cationic center and an anionic center in one molecule, namely a zwitter ion. The intermolecular salt is a compound formed by associating a cationic ion and an anionic ion, namely an ion pair.

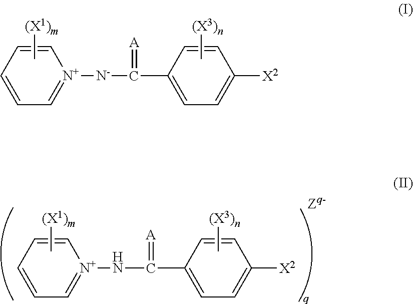

The term “unsubstituted” refers to a group consisting of a mother nucleus. In the case where only the name of a group serving as a mother nucleus is provided without accompanying the term “substituted”, this refers to “unsubstituted” unless specifically indicated otherwise.

On the other hand, the term “substituted” means that any hydrogen atom of a group serving as a mother nucleus is substituted with a group (substituent) having a structure that is identical to or different from the mother nucleus. Thus, a “substituent” is another group bound to a group serving as the mother nucleus. The number of substituents may be one or two or more. Two or more substituents may be identical to or different from each other.

The term “C1-6”, for example, indicates that the number of carbon atoms of the group serving as the mother nucleus is 1 to 6. The number of carbon atoms does not include the number of carbon atoms present in substituents. For example, a butyl group having an ethoxy group as a substituent thereof is classified as a C2 alkoxy C4 alkyl group.

There are no particular limitations on “substituents” provided that they are chemically available and achieve the effects of the present invention. Specific examples of groups that can be “substituents” include the following groups:

C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group;

C2-6 alkenyl groups such as a vinyl group, a 1-propenyl group, a 2-propenyl group (an allyl group), a 1-butenyl group, a 2-butenyl group, a 3-butenyl group, a 1-methyl-2-propenyl group, and a 2-methyl-2-propenyl group;

C2-6 alkynyl groups such as an ethynyl group, a 1-propynyl group, a 2-propynyl group, a 1-butynyl group, a 2-butynyl group, a 3-butynyl group, and a 1-methyl-2-propynyl group;

C3-8 cycloalkyl groups such as a cyclopropyl group, a cyclobutyl group, a cyclopentyl group, a cyclohexyl group, and a cubanyl group;

C6-10 aryl groups such as a phenyl group and a naphthyl group;

C6-10 aryl C1-6 alkyl groups such as a benzyl group and a phenethyl group;

3- to 6-membered heterocyclyl groups;

3- to 6-membered heterocyclyl C1-6 alkyl groups;

a hydroxy group;

C1-6 alkoxy groups such as a methoxy group, an ethoxy group, a n-propoxy group, an i-propoxy group, a n-butoxy group, a s-butoxy group, an i-butoxy group, and a t-butoxy group;

C2-6 alkenyloxy groups such as a vinyloxy group, an allyloxy group, a propenyloxy group, and a butenyloxy group;

C2-6 alkynyloxy groups such as an ethynyloxy group, and a propargyloxy group;

C6-10 aryloxy groups such as a phenoxy group, and a naphthoxy group;

C6-10 aryl C1-6 alkoxy groups such as a benzyloxy group, and a phenethyloxy group;

5- or 6-membered heteroaryloxy groups such as a thiazolyloxy group, and a pyridyloxy group;

5- or 6-membered heteroaryl C1-6 alkyloxy groups such as a thiazolylmethyloxy group, and a pyridylmethyloxy group;

a formyl group;

C1-6 alkylcarbonyl groups such as an acetyl group, and a propionyl group;

a formyloxy group;

C1-6 alkylcarbonyloxy groups such as an acetyloxy group, and a propionyloxy group;

C6-10 arylcarbonyl groups such as a benzoyl group;

C1-6 alkoxycarbonyl groups such as a methoxycarbonyl group, an ethoxycarbonyl group, a n-propoxycarbonyl group, an i-propoxycarbonyl group, a n-butoxycarbonyl group, and a t-butoxycarbonyl group;

C1-6 alkoxycarbonyloxy groups such as a methoxycarbonyloxy group, an ethoxycarbonyloxy group, a n-propoxycarbonyloxy group, an i-propoxycarbonyloxy group, a n-butoxycarbonyloxy group, and a t-butoxycarbonyloxy group;

a carboxyl group;

halogeno groups such as a fluoro group, a chloro group, a bromo group, and an iodo group;

C1-6 haloalkyl groups such as a chloromethyl group, a chloroethyl group, a trifluoromethyl group, a 1,2-dichloro-n-propyl group, and a 1-fluoro-n-butyl group;

C2-6 haloalkenyl groups such as a 2-chloro-1-propenyl group, and a 2-fluoro-1-butenyl group;

C2-6 haloalkynyl groups such as a 4,4-dichloro-1-butynyl group, a 4-fluoro-1-pentynyl group, and a 5-bromo-2-pentynyl group;

C1-6 haloalkoxy groups such as a trifluoromethoxy group, a 2-chloro-n-propoxy group, and a 2,3-dichlorobutoxy group;

C2-6 haloalkenyloxy groups such as a 2-chloropropenyloxy group, and a 3-bromobutenyloxy group;

C1-6 haloalkylcarbonyl groups such as a chloroacetyl group, a trifluoroacetyl group, and a trichloroacetyl group;

an amino group;

C1-6 alkyl-substituted amino groups such as a methylamino group, a dimethylamino group, and a diethylamino group;

C6-10 arylamino groups such as an anilino group, and a naphthylamino group;

C6-10 aryl C1-6 alkylamino groups such as a benzylamino group, and a phenethylamino group;

a formylamino group;

C1-6 alkylcarbonylamino groups such as an acetylamino group, a propanoylamino group, a butyrylamino group, and an i-propylcarbonylamino group;

C1-6 alkoxycarbonylamino groups such as a methoxycarbonylamino group, an ethoxycarbonylamino group, a n-propoxycarbonylamino group, and an i-propoxycarbonylamino group;

unsubstituted or substituted aminocarbonyl groups such as a carbamoyl group, a dimethylaminocarbonyl group, a phenylaminocarbonyl group, and a N-phenyl-N-methylaminocarbonyl group;

imino C1-6 alkyl groups such as an iminomethyl group, a (1-imino)ethyl group, and a (1-imino)-n-propyl group;

substituted or unsubstituted N-hydroxyimino C1-6 alkyl groups such as a N-hydroxy-iminomethyl group, a (1-(N-hydroxy)-imino)ethyl group, a (1-(N-hydroxy)-imino)propyl group, a N-methoxy-iminomethyl group, and a (1-(N-methoxy)-imino)ethyl group;

an aminocarbonyloxy group;

C1-6 alkyl-substituted aminocarbonyloxy groups such as an ethylaminocarbonyloxy group, and a dimethylaminocarbonyloxy group;

a mercapto group;

C1-6 alkylthio groups such as a methylthio group, an ethylthio group, a n-propylthio group, an i-propylthio group, a n-butylthio group, an i-butylthio group, a s-butylthio group, and a t-butylthio group;

C1-6 haloalkylthio groups such as a trifluoromethylthio group, and a 2,2,2-trifluoroethylthio group;

C6-10 arylthio groups such as a phenylthio group, and a naphthylthio group;

5- or 6-membered heteroarylthio groups such as a thiazolylthio group, and a pyridylthio group;

C1-6 alkylsulfinyl groups such as a methylsulfinyl group, an ethylsulfinyl group, and a t-butylsulfinyl group;

C1-6 haloalkylsulfinyl groups such as a trifluoromethylsulfinyl group, and a 2,2,2-trifluoroethylsulfinyl group;

C6-10 arylsulfinyl groups such as a phenylsulfinyl group;

5- or 6-membered heteroarylsulfinyl groups such as a thiazolylsulfinyl group, and a pyridylsulfinyl group;

C1-6 alkylsulfonyl groups such as a methylsulfonyl group, an ethylsulfonyl group, and a t-butylsulfonyl group;

C1-6 haloalkylsulfonyl groups such as a trifluoromethylsulfonyl group, and a 2,2,2-trifluoroethylsulfonyl group;

C6-10 arylsulfonyl groups such as a phenylsulfonyl group;

5- or 6-membered heteroarylsulfonyl groups such as a thiazolylsulfonyl group, and a pyridylsulfonyl group;

C1-6 alkylsulfonyloxy groups such as a methylsulfonyloxy group, an ethylsulfonyloxy group, and a t-butylsulfonyloxy group;

C1-6 haloalkylsulfonyloxy groups such as a trifluoromethylsulfonyloxy group, and a 2,2,2-trifluoroethylsulfonyloxy group;

tri C1-6 alkyl-substituted silyl groups such as a trimethylsilyl group, a triethylsilyl group, and a t-butyldimethylsilyl group;

tri C6-10 aryl-substituted silyl groups such as a triphenylsilyl group;

a pentafluorosulfanyl group;

a cyano group; and a nitro group.

Any hydrogen atom of the “substituent” may be substituted with a group having a different structure. Examples of such a substituent include C1-6 alkyl groups, C1-6 haloalkyl groups, C1-6 alkoxy groups, C1-6 haloalkoxy groups, halogeno groups, a cyano group, and a nitro group.

The “3- to 6-membered heterocyclyl group” contains, as a ring member atom, one to four hetero atoms selected from the group consisting of a nitrogen atom, an oxygen atom and a sulfur atom. The heterocyclyl group may be monocyclic or polycyclic. If at least one ring of a polycyclic heterocyclyl group is a hetero ring, the remaining rings thereof may be any of saturated alicyclic rings, unsaturated alicyclic rings and aromatic rings. Examples of the “3- to 6-membered heterocyclyl group” include 3- to 6-membered saturated heterocyclyl groups, 5- or 6-membered heteroaryl groups, and 5- or 6-membered partially unsaturated heterocyclyl groups.

Examples of the 3- to 6-membered saturated heterocyclyl groups include an aziridinyl group, an epoxy group, a pyrrolidinyl group, a tetrahydrofuranyl group, a thiazolidinyl group, a piperidyl group, a piperazinyl group, a morpholinyl group, a dioxolanyl group, and a dioxanyl group.

Examples of the 5-membered heteroaryl groups include a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, and a tetrazolyl group.

Examples of the 6-membered heteroaryl group include a pyridyl group, a pyrazinyl group, a pyrimidinyl group, a pyridazinyl group, and a triazinyl group.

### (Z^{q-})

In the formula (II), Z^{q-} indicates a counter ion, and q indicates the valence of the counter ion and is 1 or 2. Specific examples of the monovalent anion Z⁻ include Cl⁻, Br⁻, I⁻, NO³⁻, CH₃COO⁻, CH₃SO₃⁻, CF₃SO₃⁻, and TolSO₃⁻. Specific examples of the divalent anion Z²⁻ include SO₄²⁻. Tol is an abbreviation indicating an o-methylphenyl group, a m-methylphenyl group or a p-methylphenyl group.

Among these, Z^{q-} is preferably a monovalent anion Z⁻, such as Cl⁻, Br⁻, or I⁻.

### (A)

In the formulae (I) and (II), A is an oxygen atom or a sulfur atom.

In the present invention, A is preferably an oxygen atom.

### (X¹, m)

In the formulae (I) and (II), X¹ is a halogeno group, a substituted or unsubstituted C1-6 alkyl group, a substituted or unsubstituted C2-6 alkenyl group, a substituted or unsubstituted C2-6 alkynyl group, a substituted or unsubstituted C1-6 alkoxy group, a substituted or unsubstituted C2-6 alkenyloxy group, a substituted or unsubstituted C2-6 alkynyloxy group, a substituted or unsubstituted C1-6 alkylcarbonyl group, a substituted or unsubstituted C1-6 alkoxycarbonyl group, a substituted or unsubstituted C1-6 alkylthio group, a substituted or unsubstituted CT-6 alkylsulfinyl group, a substituted or unsubstituted C1-6 alkylsulfonyl group, a substituted or unsubstituted C3-8 cycloalkyl group, a substituted or unsubstituted C6-10 aryl group, a substituted or unsubstituted C6-10 aryloxy group, a substituted or unsubstituted 5- or 6-membered heteroaryl group, a substituted or unsubstituted 5- or 6-membered heteroaryloxy group, a substituted or unsubstituted C6-10 arylcarbonyl group, a substituted or unsubstituted 5- or 6-membered heteroarylcarbonyl group, a group of R^{a}R^{b}N—, a group of R^{a}R^{b}N—CO—, a group of R^{c}CO—NH—, a pentafluorosulfanyl group, a nitro group, or a cyano group.

m indicates the number of X¹ and is an integer of 0 to 5, preferably 0 or 1, and more preferably 0.

In the case where m is 2 or more, X¹ may be identical to or different from each other.

Examples of the “halogeno group” as X¹ include a fluoro group, a chloro group, a bromo group, and an iodo group.

The “C1-6 alkyl group” as X¹ may be a linear chain or a branched chain. Examples of the “C1-6 alkyl group” as X¹ include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group.

Examples of the “C2-6 alkenyl group” as X¹ include a vinyl group, a 1-propenyl group, a 2-propenyl group, a 1-butenyl group, a 2-butenyl group, a 3-butenyl group, a 1-methyl-2-propenyl group, a 2-methyl-2-propenyl group, a 1-pentenyl group, a 2-pentenyl group, a 3-pentenyl group, a 4-pentenyl group, a 1-methyl-2-butenyl group, a 2-methyl-2-butenyl group, a 1-hexenyl group, a 2-hexenyl group, a 3-hexenyl group, a 4-hexenyl group, and a 5-hexenyl group.

Examples of the “C2-6 alkynyl group” as X¹ include an ethynyl group, a 1-propynyl group, a 2-propynyl group, a 1-butynyl group, a 2-butynyl group, a 3-butynyl group, a 1-methyl-2-propynyl group, a 2-methyl-3-butynyl group, a 1-pentynyl group, a 2-pentynyl group, a 3-pentynyl group, a 4-pentynyl group, a 1-methyl-2-butynyl group, a 2-methyl-3-pentynyl group, a 1-hexynyl group, and a 1,1-dimethyl-2-butynyl group.

Examples of the “C1-6 alkoxy group” as X¹ include a methoxy group, an ethoxy group, a n-propoxy group, a n-butoxy group, a n-pentyloxy group, a n-hexyloxy group, an i-propoxy group, an i-butoxy group, a s-butoxy group, a t-butoxy group, and an i-hexyloxy group.

Examples of the “C2-6 alkenyloxy group” as X¹ include a vinyloxy group, an allyloxy group, a propenyloxy group, and a butenyloxy group.

Examples of the “C2-6 alkynyloxy group” as X¹ include an ethynyloxy group, and a propargyloxy group.

Examples of the “C1-6 alkylcarbonyl group” as X¹ include an acetyl group, and a propionyl group.

Examples of the “C1-6 alkoxycarbonyl group” as X¹ include a methoxycarbonyl group, an ethoxycarbonyl group, a n-propoxycarbonyl group, an i-propoxycarbonyl group, and a t-butoxycarbonyl group.

Examples of the “C1-6 alkylthio group” as X¹ include a methylthio group, an ethylthio group, a n-propylthio group, a n-butylthio group, a n-pentylthio group, a n-hexylthio group, and an i-propylthio group.

Examples of the “C1-6 alkylsulfinyl group” as X¹ include a methylsulfinyl group, an ethylsulfinyl group, and a t-butylsulfinyl group.

Examples of the “C1-6 alkylsulfonyl group” as X¹ include a methylsulfonyl group, an ethylsulfonyl group, and a t-butylsulfonyl group.

Preferable examples of a substituent on the “C1-6 alkyl group”, “C2-6 alkenyl group”, “C2-6 alkynyl group”, “C1-6 alkoxy group”, “C2-6 alkenyloxy group”, “C2-6 alkynyloxy group”, “C1-6 alkylcarbonyl group”, “CT-6 alkoxycarbonyl group”, “C1-6 alkylthio group”, “C1-6 alkylsulfinyl group”, or “C1-6 alkylsulfonyl group” as X¹ include: halogeno groups, such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group, and a naphthyl group; C6-10 aryl groups substituted with a halogeno group, a C1-6 haloalkyl group, or a C1-6 haloalkoxy group, such as a 4-chlorophenyl group, a 4-trifluoromethylphenyl group, and a 4-trifluoromethoxyphenyl group; and a cyano group.

Examples of the “C3-8 cycloalkyl group” as X¹ include a cyclopropyl group, a cyclobutyl group, a cyclopentyl group, a cyclohexyl group, and a cycloheptyl group.

Examples of the “C6-10 aryl group” as X¹ include a phenyl group, and a naphthyl group.

Examples of the “C6-10 aryloxy group” as X¹ include a phenoxy group, and a naphthoxy group.

The “5- or 6-membered heteroaryl group” as X¹ contains, as a ring member atom, one, two, three, or four hetero atoms selected from the group consisting of a nitrogen atom, an oxygen atom and a sulfur atom. In the case where at least two hetero atoms are present, the hetero atoms may be identical to or different from each other.

Examples of the 5-membered heteroaryl groups include a pyrrolyl group, a furyl group, a thienyl group, an imidazolyl group, a pyrazolyl group, an oxazolyl group, an isoxazolyl group, a thiazolyl group, an isothiazolyl group, a triazolyl group, an oxadiazolyl group, a thiadiazolyl group, and a tetrazolyl group. Examples of the 6-membered heteroaryl group include a pyridyl group, a pyrazinyl group, a pyrimidinyl group, a pyridazinyl group, and a triazinyl group.

The “5- or 6-membered heteroaryloxy group” as X¹ has a structure in which a 5- or 6-membered heteroaryl group and an oxy group are bonded. Specific examples thereof include a thiazolyloxy group, and a pyridyloxy group;

The “C6-10 arylcarbonyl group” as X¹ has a structure in which a C6-10 aryl group and a carbonyl group are bonded. Specific examples thereof include a benzoyl group.

The “5- or 6-membered heteroarylcarbonyl group” as X¹ has a structure in which a 5- or 6-membered heteroaryl group and a carbonyl group are bonded. Specific examples thereof include a thiazolylcarbonyl group and a pyridylcarbonyl group.

Preferable examples of a substituent on the “C3-8 cycloalkyl group”, “C6-10 aryl group”, “C6-10 aryloxy group”, “5- or 6-membered heteroaryl group”, “5- or 6-membered heteroaryloxy group”, “C6-10 arylcarbonyl group”, or “5- or 6-membered heteroarylcarbonyl group” as X¹ include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group, and a naphthyl group; C6-10 aryl groups substituted with a halogeno group, a C1-6 haloalkyl group, or a C1-6 haloalkoxy group, such as a 4-chlorophenyl group, a 4-trifluoromethylphenyl group, and a 4-trifluoromethoxyphenyl group; and a cyano group.

In the “R^{a}R^{b}N—” or “R^{a}R^{b}N—CO—” as X¹, R^{a} is a hydrogen atom, a substituted or unsubstituted C1-6 alkyl group, or a substituted or unsubstituted C6-10 aryl group, and R^{b} is a hydrogen atom, a substituted or unsubstituted C1-6 alkyl group, or a substituted or unsubstituted C6-10 aryl group.

The “C1-6 alkyl group” as R^{a} or R^{b} may be a linear chain or a branched chain. Examples of the “C1-6 alkyl group” as R^{a} or R^{b} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group. Preferable examples of a substituent on the “C1-6 alkyl group” include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group and a naphthyl group; C6-10 aryl groups substituted with a halogeno group, a C1-6 haloalkyl group or a C1-6 haloalkoxy group, such as a 4-chlorophenyl group, a 4-trifluoromethylphenyl group, and a 4-trifluoromethoxyphenyl group; and a cyano group.

Examples of the “C6-10 aryl group” as R^{a} or R^{b} include a phenyl group, and a naphthyl group. Preferable examples of a substituent on the “C6-10 aryl group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

In the “R^{c}CO—NH—” as X¹, R^{c} is a hydrogen atom, a substituted or unsubstituted C1-6 alkyl group, or a substituted or unsubstituted C6-10 aryl group.

The “C1-6 alkyl group” as R^{c} may be a linear chain or a branched chain. Examples of the “C1-6 alkyl group” as R^{c} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group. Preferable examples of a substituent on the “C1-6 alkyl group” include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group and a naphthyl group; C6-10 aryl groups substituted with a halogeno group, a C1-6 haloalkyl group or a C1-6 haloalkoxy group, such as a 4-chlorophenyl group, a 4-trifluoromethylphenyl group, and a 4-trifluoromethoxyphenyl group; and a cyano group.

Examples of the “C6-10 aryl group” as R^{c} include a phenyl group and a naphthyl group. Preferable examples of a substituent on the “C6-10 aryl group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

X¹ is preferably a halogeno group, an unsubstituted C1-6 alkyl group, a halogeno group-substituted C1-6 alkyl group, an unsubstituted C1-6 alkoxy group, or a halogeno group-substituted C1-6 alkoxy group, and more preferably an unsubstituted C1-6 alkyl group.

Examples of the “halogeno group-substituted C1-6 alkyl group” (C1-6 haloalkyl group)” as X¹ include: a difluoromethyl group, a trifluoromethyl group, a 2,2,2-trifluoroethyl group, a 2,2,2,1,1-pentafluoroethyl group.

Examples of the “halogeno group-substituted C1-6 alkoxy group” (C1-6 haloalkoxy group) as X¹ include: a difluoromethoxy group, a trifluoromethoxy group, a 2,2-difluoroethoxy group, a 2,2,2-trifluoroethoxy group, a 2,2,1,1-tetrafluoroethoxy group, and a 2,2,2,1,1-pentafluoroethoxy group.

### (X²)

X² is a C5-8 alkyl group; a C2-8 haloalkyl group; a substituted or unsubstituted C6-10 aryl-substituted C1-6 alkyl group; a substituted or unsubstituted 5- or 6-membered heteroaryl-substituted C1-6 alkyl group; a substituted or unsubstituted C6-10 aryloxy-substituted C1-6 alkyl group; a substituted or unsubstituted 5- or 6-membered heteroarylox-substituted C1-6 alkyl group; a C3-6 alkenyl group; a C2-6 haloalkenyl group; a substituted or unsubstituted C6-10 aryl-substituted C2-6 alkenyl group; a substituted or unsubstituted 5- or 6-membered heteroaryl-substituted C2-6 alkenyl group; a C2-6 alkynyl group; a C2-6 haloalkynyl group; a substituted or unsubstituted C6-10 aryl-substituted C2-6 alkynyl group; a substituted or unsubstituted 5- or 6-membered heteroaryl-substituted C2-6 alkynyl group; a hydroxy group; a substituted C1-8 alkoxy group; a substituted or unsubstituted C2-6 alkenyloxy group; a substituted or unsubstituted C2-6 alkynyloxy group; a C2-6 alkylcarbonyl group; a substituted C1-6 alkylcarbonyl group; a substituted or unsubstituted C1-6 alkoxycarbonyl group; a substituted or unsubstituted C1-6 alkylthio group; a substituted or unsubstituted C1-6 alkylsulfinyl group; a substituted or unsubstituted C1-6 alkylsulfonyl group; a substituted or unsubstituted C3-8 cycloalkyl group; a substituted or unsubstituted C6-10 aryloxy group; a substituted or unsubstituted 5- or 6-membered heteroaryloxy group; a substituted or unsubstituted C6-10 arylcarbonyl group; a substituted or unsubstituted 5- or 6-membered heteroarylcarbonyl group; a group of R^{a1}R^{b1}N—; a carbamoyl group; a group of R^{a1}R^{b1}N—CO—; a group of R^{c1}CO—N(R^{d1})—; a group of R^{a1}R^{b1}N—CO—N(R^{d1})—; a group of R^{a1}R^{b1}N—CS—N(R^{a1})—; a group of R^{e1}O—N═CR^{f1}—; a group of R^{g1}SO₂—N(R^{a1})—; a group of R^{g1}SO₂NH—N═CR^{f1}—; or a pentafluorosulfanyl group.

The “C5-8 alkyl group” as X² may be a linear chain or a branched chain. Examples of the “C5-8 alkyl group” as X² include a n-pentyl group, a n-hexyl group, a n-octyl group, an i-pentyl group, a neopentyl group, a 2-methylbutyl group, a 2,2-dimethylpropyl, and an i-hexyl group.

Examples of the “C2-8 haloalkyl group” as X² include a 2,2,2-trifluoroethyl group.

Examples of the “C6-10 aryl C1-6 alkyl group” as X² include a benzyl group, and a phenethyl group.

Examples of the “5- or 6-membered heteroaryl C1-6 alkyl group” as X² include a thiazolylmethyl group, and a pyridylmethyl group.

Examples of the “C6-10 aryloxy C1-6 alkyl group” as X² include a phenoxymethyl group.

Examples of the “5- or 6-membered heteroaryloxy C1-6 alkyl group” as X² include a pyridyloxymethyl group.

Examples of the “C3-6 alkenyl group” as X² include a 1-propenyl group, a 2-propenyl group (an allyl group), a 1-butenyl group, a 2-butenyl group, a 3-butenyl group, a 1-methyl-2-propenyl group, and a 2-methyl-2-propenyl group.

Examples of the “C2-6 haloalkenyl group” as X² include a 2-chloro-1-propenyl group, a 2-fluoro-1-butenyl group, and a 3,3,3-trifluoro-1-propenyl group.

Examples of the “C6-10 aryl C2-6 alkenyl group” as X² include a styryl group, and a phenylprop-2-en-1-yl group.

Examples of the “5- or 6-membered heteroaryl C2-6 alkenyl group” as X² include a pyridylvinyl group, and a pyridylprop-2-en-1-yl group.

Examples of the “C2-6 alkynyl group” as X² include an ethynyl group, a 1-propynyl group, a 2-propynyl group, a 1-butynyl group, a 2-butynyl group, a 3-butynyl group, and a 1-methyl-2-propynyl group.

Examples of the “C2-6 haloalkynyl group” as X² include a 3,3,3-trifluoropropa-1-yn-1-yl group, a 4,4-dichloro-1-butynyl group, a 4-fluoro-1-pentynyl group, and a 5-bromo-2-pentynyl group.

Examples of the “C6-10 aryl C2-6 alkynyl group” as X² include a phenylethynyl group.

Examples of the “5- or 6-membered heteroaryl C2-6 alkynyl group” as X² include a pyridylethynyl group.

Examples of the “C2-6 alkenyloxy group” as X² include the same groups as those mentioned above as X¹.

Examples of a substituent on the “C2-6 alkenyloxy” as X² include the same groups as those mentioned above as X¹, and halogeno groups such as a fluoro group, a chloro group, and a bromo group are preferable.

Examples of the “C2-6 alkylcarbonyl group” as X² include a propionyl group.

Preferable examples of a substituent on the “substituted C1-8 alkoxy group” or “substituted C1-6 alkylcarbonyl group” as X² include: halogeno groups, such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C1-6 haloalkoxy C1-6 haloalkoxy groups such as a 1,1,2,2,3,3-hexafluoro-3-trifluoromethoxy-propoxy group; C1-6 alkylthio groups such as a methylthio group; C1-6 haloalkylthio groups such as a trifluoromethylthio group; C6-10 aryl groups such as a phenyl group, and a naphthyl group; C6-10 aryl groups substituted with a halogeno group, a C1-6 haloalkyl group, or a C1-6 haloalkoxy group, such as a 4-chlorophenyl group, a 4-trifluoromethylphenyl group, and a 4-trifluoromethoxyphenyl group; and a cyano group, and more preferable examples thereof include halogeno groups; C1-6 haloalkoxy groups; C1-6 haloalkoxy C1-6 haloalkoxy groups; C1-6 haloalkylthio groups; and C6-10 aryl groups which may be substituted with a halogeno group, a C1-6 haloalkyl group, or a C1-6 haloalkoxy group. In the “substituted C1-8 alkoxy group”, at least one substituent selected from the above-mentioned substituents may be substituted.

Examples of the “C1-6 alkylthio group”, “C1-6 alkylsulfinyl group”, or “C1-6 alkylsulfonyl group” as X² include the same groups as those mentioned as X¹.

Examples of a substituent on the “C1-6 alkylthio group”, “C1-6 alkylsulfinyl group”, or “C1-6 alkylsulfonyl group” as X² include the same groups as those mentioned as X¹, and halogeno groups, such as a fluoro group, a chloro group, a bromo group, and an iodo group are preferable.

Examples of the “C3-8 cycloalkyl group” as X² include the same groups as those mentioned as X¹, and a cyclopropyl group is preferable.

Examples of a substituent on the “C3-8 cycloalkyl group” as X² include the same groups as those mentioned as X¹, and preferable examples thereof include: halogeno groups, such as a fluoro group, a chloro group, a bromo group, and an iodo group; C1-6 haloalkyl groups such as a chloromethyl group, a chloroethyl group, a trifluoromethyl group, a 1,2-dichloro-n-propyl group, and a 1-fluoro-n-butyl group; C1-6 haloalkoxy groups such as a 2-chloro-n-propoxy group, a 2,3-dichlorobutoxy group, and a trifluoromethoxy group.

Examples of the “C6-10 aryloxy group” as X² include the same groups as those mentioned as X¹, and a phenoxy group is preferable.

Examples of the “5- or 6-membered heteroaryloxy group” as X² include the same groups as those mentioned as X¹, and a pyridyloxy group is preferable.

Examples of a substituent on the “5- or 6-membered heteroaryloxy group” as X² include the same groups as those mentioned as X¹, and preferable examples thereof include: halogeno groups such as a fluoro group, a chloro group, and a bromo group; and C1-6 haloalkyl groups such as a chloromethyl group, a chloroethyl group, a trifluoromethyl group, a 1,2-dichloro-n-propyl group, and a 1-fluoro-n-butyl group.

Examples of the “C6-10 arylcarbonyl group” as X² include a benzoyl group.

Examples of a substituent on the “C6-10 arylcarbonyl group” as X² include C1-6 haloalkoxy groups such as a 2-chloro-n-propoxy group, a 2,3-dichlorobutoxy group, and a trifluoromethoxy group.

Preferable examples of a substituent on the “C6-10 aryl C1-6 alkyl group”, “5- or 6-membered heteroaryl C1-6 alkyl group”, “C6-10 aryloxy group”, “C6-10 aryloxy C1-6 alkyl group”, “5- or 6-membered heteroaryloxy C1-6 alkyl group”, “C6-10 aryl C2-6 alkenyl group”, “5- or 6-membered heteroaryl C2-6 alkenyl group”, “C6-10 aryl C2-6 alkynyl group”, or “5- or 6-membered heteroaryl C2-6 alkynyl group” as X² include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group, and more preferable examples thereof include C1-6 haloalkyl groups and C1-6 haloalkoxy groups.

In the “R^{a1}R^{b1}N—” “R^{a1}R^{b1}N—CO—”, “R^{a1}R^{b1}N—CO—N(R^{d1})—”, or “R^{a1}R^{b1}N—CS—N(R^{d1})—” as X², R^{a1} is a substituted or unsubstituted C1-6 alkyl group, or a substituted or unsubstituted C6-10 aryl group, R^{b1} is a hydrogen atom, a substituted or unsubstituted C1-6 alkyl group, or a substituted or unsubstituted C6-10 aryl group, and R^{d1} is a hydrogen atom, a substituted or unsubstituted C1-6 alkyl group, or a substituted or unsubstituted C6-10 aryl group.

The “C1-6 alkyl group” as R^{a1} or R^{b1} may be a linear chain or a branched chain. Examples of the “C1-6 alkyl group” as R^{a1} or R^{b1} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group. Preferable examples of a substituent on the “C1-6 alkyl group” include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C1-6 haloalkylthio groups such as a trifluoromethylthio group; C6-10 aryl groups such as a phenyl group and a naphthyl group; C6-10 aryl groups substituted with a halogeno group, a C1-6 haloalkyl group or a C1-6 haloalkoxy group, such as a 4-chlorophenyl group, a 4-trifluoromethylphenyl group, and a 4-trifluoromethoxyphenyl group; and a cyano group, and more preferable examples thereof include halogeno groups; C1-6 haloalkylthio groups; and C6-10 aryl groups substituted with a C1-6 haloalkoxy group.

Examples of the “C6-10 aryl group” as R^{a1} or R^{b1} include a phenyl group and a naphthyl group, and a phenyl group is preferable. Preferable examples of a substituent on the “C6-10 aryl group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group, and more preferable examples thereof include: halogeno groups; C1-6 haloalkyl groups; and C1-6 haloalkoxy groups.

In the “R^{a1}R^{b1}N—”, “R^{a1}R^{b1}N—CO—”, “R^{a1}R^{b1}N—CO—N(R^{d1})—”, or “R^{a1}R^{b1}N—CS—N(R^{d1})—” as X², R^{a1} and R^{b1} may be bonded together to form a divalent organic group.

Examples of the formable divalent organic group include substituted or unsubstituted C2-6 alkylene groups.

Examples of the “C2-6 alkylene group” include a dimethylene group, a trimethylene group, and a tetramethylene group. Preferable examples of a substituent on the “C2-6 alkylene group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, and a t-butyl group; and C1-6 haloalkyl groups such as a chloromethyl group, a chloroethyl group, a trifluoromethyl group, a 1,2-dichloro-n-propyl group, and a 1-fluoro-n-butyl group.

The “C1-6 alkyl group” as R^{d1} may be a linear chain or a branched chain. Examples of the “C1-6 alkyl group” as R^{d1} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group. Preferable examples of a substituent on the “C1-6 alkyl group” include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group and a naphthyl group; C6-10 aryl groups substituted with a halogeno group, a C1-6 haloalkyl group or a C1-6 haloalkoxy group, such as a 4-chlorophenyl group, a 4-trifluoromethylphenyl group, and a 4-trifluoromethoxyphenyl group; and a cyano group.

Examples of the “C6-10 aryl group” as R^{d} include a phenyl group, and a naphthyl group. Preferable examples of a substituent on the “C6-10 aryl group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

In the “R^{a1}R^{b1}N—”, “R^{a1}R^{b1}N—CO—”, “R^{a1}R^{b1}N—CO—N(R^{d1})—”, or “R^{a1}R^{b1}N—CS—N(R^{d1})—” as X², R^{b1} is preferably a hydrogen atom or a C1-6 alkyl group.

In the “R^{a1}R^{b1}N—CO—N(R^{d1})—” or “R^{a1}R^{b1}N—CS—N(R^{d1})—” as X², R^{d1} is preferably a hydrogen atom.

In the “R^{c1}CO—N(R^{d1})—” as X², R^{c1} is preferably a hydrogen atom, a substituted or unsubstituted C1-6 alkyl group, a substituted or unsubstituted C1-6 alkoxy group, or a substituted or unsubstituted C6-10 aryl group, and R^{d1} is the group as mentioned above.

The “C1-6 alkyl group” as R^{c1} may be a linear chain or a branched chain. Examples of the “C1-6 alkyl group” as R^{c1} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group. Preferable examples of a substituent on the “C1-6 alkyl group” include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group and a naphthyl group; C6-10 aryl groups substituted with a halogeno group, a C1-6 haloalkyl group or a C1-6 haloalkoxy group, such as a 4-chlorophenyl group, a 4-trifluoromethylphenyl group, and a 4-trifluoromethoxyphenyl group; and a cyano group

Examples of the “C6-10 aryl group” as R^{c} include a phenyl group, and a naphthyl group, and a phenyl group is preferable. Preferable examples of a substituent on the “C6-10 aryl group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

Examples of the “C1-6 alkoxy group” as R^{c1} include a methoxy group, an ethoxy group, a n-propoxy group, a n-butoxy group, a n-pentyloxy group, a n-hexyloxy group, an i-propoxy group, an i-butoxy group, a s-butoxy group, a t-butoxy group, and an i-hexyloxy group. Preferable examples of a substituent on the “C1-6 alkoxy group” include: halogeno groups, such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group, and a naphthyl group; C6-10 aryl groups substituted with a halogeno group, a C1-6 haloalkyl group, or a C1-6 haloalkoxy group, such as a 4-chlorophenyl group, a 4-trifluoromethylphenyl group, and a 4-trifluoromethoxyphenyl group; and a cyano group.

Among these, in the “R^{c1}CO—N(R^{d1})—” as X², R^{c1} is preferably a C1-6 alkoxy group; or a C6-10 aryl group substituted with a C1-6 haloalkyl group, and R^{d1} is preferably a hydrogen atom; a C1-6 alkyl group; or a C1-6 alkyl group substituted with a C6-10 aryl group substituted with a C1-6 haloalkoxy group.

In the “R^{e1}O—N═CR^{f1}—” as X², R^{e1} is a hydrogen atom, a substituted or unsubstituted C1-6 alkyl group, or a substituted or unsubstituted C6-10 aryl group, and R^{f1} is a hydrogen atom, or a substituted or unsubstituted C1-6 alkyl group.

The “C1-6 alkyl group” as R^{e1} or R^{f1} may be a linear chain or a branched chain. Examples of the “C1-6 alkyl group” as R^{e1} or R^{f1} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group. Preferable examples of a substituent on the “C1-6 alkyl group” include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group and a naphthyl group; C6-10 aryl groups substituted with a halogeno group, a C1-6 haloalkyl group or a C1-6 haloalkoxy group, such as a 4-chlorophenyl group, a 4-trifluoromethylphenyl group, and a 4-trifluoromethoxyphenyl group; and a cyano group, and more preferable examples thereof include halogeno groups.

Examples of the “C6-10 aryl group” as R^{e1} include a phenyl group, and a naphthyl group. Preferable examples of a substituent on the “C6-10 aryl group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

In the “R^{e1}O—N═CR^{f1}−” as X², R^{e1} is preferably a hydrogen atom or a C1-6 alkyl group, and R^{f1} is preferably a C1-6 haloalkyl group.

In the “R^{g1}SO₂—N(R^{d1})—” or “R^{g1}SO₂NH—N═CR^{f1}—” as X², R^{g1} is a substituted or unsubstituted C1-6 alkyl group, or a substituted or unsubstituted C6-10 aryl group, and R^{d1} and R^{f1} are the groups as mentioned above, respectively.

The “C1-6 alkyl group” as R^{g1} may be a linear chain or a branched chain. Examples of the “C1-6 alkyl group” as R^{g1} include a methyl group, an ethyl group, a n-propyl group, a n-butyl group, a n-pentyl group, a n-hexyl group, an i-propyl group, an i-butyl group, a s-butyl group, a t-butyl group, an i-pentyl group, a neopentyl group, a 2-methylbutyl group, a 2,2-dimethylpropyl group, and an i-hexyl group. Preferable examples of a substituent on the “C1-6 alkyl group” include: halogeno groups such as a fluoro group, a chloro group, a bromo group, and an iodo group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; C6-10 aryl groups such as a phenyl group and a naphthyl group; C6-10 aryl groups substituted with a halogeno group, a C1-6 haloalkyl group or a C1-6 haloalkoxy group, such as a 4-chlorophenyl group, a 4-trifluoromethylphenyl group, and a 4-trifluoromethoxyphenyl group; and a cyano group.

Examples of the “C6-10 aryl group” as R^{g1} include a phenyl group, and a naphthyl group, and a phenyl group is preferable. Preferable examples of a substituent on the “C6-10 aryl group” include: halogeno groups, such as a fluoro group, a chloro group, a bromo group, and an iodo group; C1-6 alkyl groups such as a methyl group, an ethyl group, a n-propyl group, an i-propyl group, a n-butyl group, a s-butyl group, an i-butyl group, a t-butyl group, a n-pentyl group, and a n-hexyl group; C1-6 haloalkyl groups such as a chloromethyl group, a chloroethyl group, a trifluoromethyl group, a 1,2-dichloro-n-propyl group, and a 1-fluoro-n-butyl group; a hydroxy group; C1-6 alkoxy groups such as a methoxy group, an ethoxy group, a n-propoxy group, an i-propoxy group, a n-butoxy group, a s-butoxy group, an i-butoxy group, and a t-butoxy group; C1-6 haloalkoxy groups such as a 2-chloro-n-propoxy group, a 2,3-dichlorobutoxy group, and a trifluoromethoxy group; and a cyano group.

Among these, in the “R^{g1}SO₂—N(R^{d1})—” or “R^{g1}SO₂NH—N═CR^{f1}—” as X², R^{g1} is preferably a C6-10 aryl group which may be substituted with a C1-6 alkyl group or a C1-6 haloalkyl group, and R^{d1} and R^{f1} are preferably hydrogen atoms.

Specific examples of other groups as X² include the same groups as those mentioned as X¹.

Among these, X² is preferably: a C2-8 haloalkyl group; a substituted or unsubstituted C6-10 aryl-substituted C1-6 alkyl group; a substituted or unsubstituted C6-10 aryloxy-substituted C1-6 alkyl group; a C2-6 haloalkenyl group; a substituted or unsubstituted C6-10 aryl-substituted C2-6 alkenyl group; a C2-6 haloalkynyl group; a substituted or unsubstituted C6-10 aryl-substituted C2-6 alkynyl group; a substituted C1-8 alkoxy group; a substituted or unsubstituted C2-6 alkenyloxy group; a substituted or unsubstituted C2-6 alkynyloxy group; a substituted or unsubstituted C1-6 alkoxycarbonyl group; a substituted or unsubstituted C1-6 alkylthio group; a substituted or unsubstituted C1-6 alkylsulfinyl group; a substituted or unsubstituted C1-6 alkylsulfonyl group; a substituted or unsubstituted C3-8 cycloalkyl group; a substituted or unsubstituted C6-10 aryloxy group; a substituted or unsubstituted 5- or 6-membered heteroaryloxy group; a substituted or unsubstituted C6-10 arylcarbonyl group; a group of R^{a1}R^{b1}N—; a group of R^{a1}R^{b1}N—CO—; a group of R^{c1}CO—N(R^{d1})—; a group of R^{a1}R^{b1}N—CO—N(R^{d1})—; a group of R^{a1}R^{b1}N—CS—N(R^{d1})—; a group of R^{e1}O—N═CR^{f1}—; a group of R^{g1}SO₂—N(R^{d1})—; or a group of R^{g1}SO₂NH—N═CR^{f1}—, and more preferably a C2-8 haloalkyl group; a substituted or unsubstituted C6-10 aryl-substituted C1-6 alkyl group; a substituted or unsubstituted C6-10 aryloxy-substituted C1-6 alkyl group; a C2-6 haloalkenyl group; a substituted or unsubstituted C6-10 aryl-substituted C2-6 alkenyl group; a substituted or unsubstituted C6-10 aryl-substituted C2-6 alkynyl group; a substituted C1-8 alkoxy group; a substituted or unsubstituted C1-6 alkylthio group; a substituted or unsubstituted C1-6 alkylsulfonyl group; a substituted or unsubstituted C3-8 cycloalkyl group; a substituted or unsubstituted C6-10 aryloxy group; a substituted or unsubstituted 5- or 6-membered heteroaryloxy group; a substituted or unsubstituted C6-10 arylcarbonyl group; or a group of R^{e1}O—N═CR^{f1}—.

### (X³, n)

X³ is a halogeno group, a substituted or unsubstituted C1-6 alkyl group, a hydroxy group, a substituted or unsubstituted C1-6 alkoxy group, a substituted or unsubstituted C1-6 alkylthio group, a substituted or unsubstituted C1-6 alkylsulfinyl group, a substituted or unsubstituted C1-6 alkylsulfonyl group, a pentafluorosulfanyl group, a nitro group, or a cyano group. n indicates the number of X³ and is an integer of 0 to 4.

Specific examples of these groups as X³ include the same groups as those mentioned as X¹. In the case where n is 2 or more, X³ may be identical to or different from each other.

Among these, X³ is preferably a halogeno group, and n is preferably an integer of 0 to 2.

The pyridinium salt according to the present invention is not particularly limited by the preparation method thereof. For example, the pyridinium salt according to the present invention (hereinafter, may be referred to as a “compound according to the present invention”) may be obtained utilizing a known reaction mentioned in examples or the like.

The compound according to the present invention may be obtained, for example, by the following method.

### (Preparation of N-Aminopyridinium Salt)

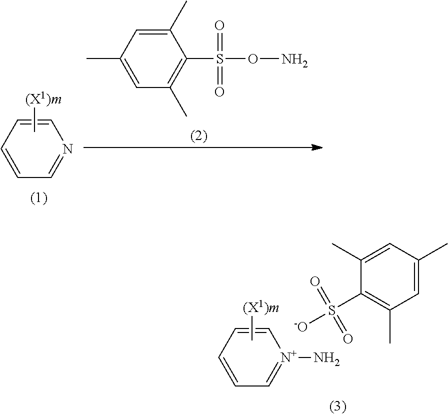

A pyridine compound (1) and an O-(mesitylsulfonyl)hydroxylamine (2) are reacted to obtain a N-aminopyridinium salt (3).

A hydroxylamine O-sulfonic acid or a O-(diphenylphosphinyl)hydroxylamine may also be used instead of the O-(mesitylsulfonyl)hydroxylamine.

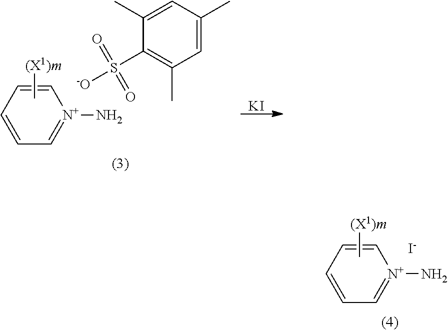

Although the N-aminopyridinium salt (3) may be directly condensed with a carboxylic acid compound (5) successively, the N-aminopyridinium salt (3) may be subjected to salt-exchange to obtain a N-aminopyridinium salt (4) which is an iodide salt, in the case where the stability of the substrate is required to be considered.

In the reaction formula, X¹ and m are the same as those in the formula (I) and formula (II).

### (Condensation Reaction)

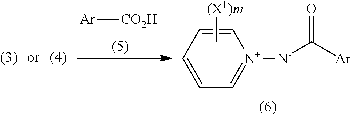

The N-aminopyridinium salt (3) or (4) and the carboxylic acid compound (5) are condensed in the presence of a condensation agent available to synthesize amides, thereby obtaining a compound (6) according to the present invention.

Examples of the condensation agent include DCC (dicyclohexylcarbodiimide), DIC (diisopropylcarbodiimide), and EDI (1-ethyl-3-(3-dimethylaminopropyl)-carbodiimide).

In the reaction formula, Ar is a benzene ring moiety having X² and (X³)n in the formula (I) and formula (II). (A) substituent(s) on the benzene ring may be appropriately replaced after the condensation reaction.

In the case where a compound in which A in the formula (I) or formula (II) is sulfur is prepared, the compound may be obtained by using a dithiocarboxylic acid ester (7) instead of the carboxylic acid compound (5).

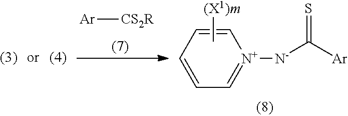

A base such as potassium carbonate or sodium ethoxide is preferably used in the condensation reaction with the dithiocarboxylic acid ester (7).

In the reaction formula, R is a C1-6 alkyl group such as a methyl group or an ethyl group.

The compound according to the present invention exhibits excellent control effects against pests, such as various agricultural insect pests or acarians which affect plant growth.

In addition, the compound according to the present invention is a highly safe substance as it exhibits low phytotoxicity against crop plants and low toxicity to fish and warm-blooded animals. Thus, the compound according to the present invention is useful as an active ingredient of an insecticide or acaricide.

In addition, in recent years, resistance to various existing drugs has increased in many insect pests such as plutellidae, delphacidae, cicadellidae, and aphids, the problem of the lack of efficacy of these drugs has arisen, and thus drugs effective against insect pests in resistant strains are also desired. The compound according to the present invention has excellent control effects not only on susceptible strains but also on insect pests of various resistant strains and even acarians of acaricide-resistant strains.

The compound according to the present invention has excellent control effects against ectoparasites and endoparasites that cause harm to humans or livestock. In addition, the compound according to the present invention is a highly safe substance as it has low toxicity to fish and warm-blooded animals. Thus, the compound according to the present invention is useful as an active ingredient of an ectoparasite-control agent or an endoparasite-control agent.

The compound according to the present invention exhibits efficacy in all developmental stages of organisms to be controlled, and exhibits excellent control effects against eggs, nymphs, larvae, pupae, and adults of acarians or insects, for example.

### (Pest Control Agent, Insecticide or Acaricide)

A pest control agent, insecticide or acaricide according to the present invention contains at least one selected from the compounds according to the present invention, as an active ingredient thereof. Although the amount of the compound according to the present invention contained in the pest control agent, insecticide or acaricide according to the present invention is not particularly limited, provided that control effects are exhibited against pests, agricultural insect pests or acarians, the amount thereof relative to 100 parts by mass of the pest control agent, insecticide or acaricide according to the present invention is generally preferably 0.01 parts by mass to 90 parts by mass, and more preferably 0.1 parts by mass to 50 parts by mass.

It is preferable that the pest control agent, insecticide or acaricide according to the present invention be applied to cereals; vegetables; root vegetables; potatoes; flowers and ornamental plants; fruit-bearing trees; foliage plants; trees, such as tea, coffee, or cacao; feed crops; lawn grasses; or plants such as cotton.

In the application to plants, the pest control agent, insecticide or acaricide according to the present invention may be applied to any portions, such as leaves, stems, stalks, flowers, buds, fruits, seeds, sprouts, roots, tubers, tuberous roots, shoots, or slips.

The pest control agent, insecticide or acaricide according to the present invention is not particularly limited by the species of plant to which it is applied. Examples of the plant species include original species, varieties, improved varieties, cultivars, mutants, hybrid bodies, and gene recombinants (GMO).

The pest control agent according to the present invention may be used to control various agricultural insect pests and acarians by conducting seed treatment, foliage application, soil application, or submerged application.

Specific examples of agricultural insect pests and acarians to be controlled with the pest control agent according to the present invention are shown below.

(1) Butterflies and moths of the order Lepidoptera

(a) Moths belonging to the family Arctiidae, such as Hyphantria cunea, and Lemyra imparilis;
(b) Moths belonging to the family Bucculatricidae, such as Bucculatrix pyrivorella;
(c) Moths belonging to the family Carposinidae, such as Carposina sasakii;
(d) Moths belonging to the family Crambidae, such as Diaphania indica, and Diaphania nitidalis, of Diaphania spp.; Ostrinia furnacalis, Ostrinia nubilalis, and Ostrinia scapulalis, of Ostrinia spp.; and others such as Chilo suppressalis, Cnaphalocrocis medinalis, Conogethes punctiferalis, Diatraea grandiosella, Glyphodes pyloalis, Hellula undalis, and Parapediasia teterrella;
(e) Moths belonging to the family Gelechiidae, such as Helcystogramma triannulella, Pectinophora gossypiella, Phthorimaea operculella, and Sitotroga cerealella;
(f) Moths belonging to the family Geometridae, such as Ascotis selenaria;
(g) Moths belonging to the family Gracillariidae, such as Caloptilia theivora, Phyllocnistis citrella, and Phyllonorycter ringoniella;
(h) Butterflies belonging to the family Hesperiidae, such as Parnara guttata;
(i) Moths belonging to the family Lasiocampidae, such as Malacosoma neustria;
(j) Moths belonging to the family Lymantriidae, such as Lymantria dispar, and Lymantria monacha, of Lymantria spp.; and others such as Euproctis pseudoconspersa, and Orgyia thyellina;
(k) Moths belonging to the family Lyonetiidae, such as Lyonetia clerkella, and Lyonetia prunifoliella malinella, of Lyonetia spp.;
(l) Moths belonging to the family Noctuidae, such as Spodoptera depravata, Spodoptera eridania, Spodoptera exigua, Spodoptera frugiperda, Spodoptera littoralis, and Spodoptera litura, of Spodoptera spp.; Autographa gamma, and Autographa nigrisigna, of Autographa spp.; Agrotis ipsilon, and Agrotis segetum, of Agrotis spp.; Helicoverpa armigera, Helicoverpa assulta, and Helicoverpa zea, of Helicoverpa spp.; Heliothis armigera, and Heliothis virescens, of Heliothis spp.; and others such as Aedia leucomelas, Ctenoplusia agnata, Eudocima tyrannus, Mamestra brassicae, Mythimna separata, Naranga aenescens, Panolis japonica, Peridroma saucia, Pseudoplusia includens, and Trichoplusia ni;
(m) Moths belonging to the family Nolidae, such as Earias insulana;
(n) Butterflies belonging to the family Pieridae, such as Pieris brassicae, and Pieris rapae crucivora, of Pieris spp.;
(o) Moths belonging to the family Plutellidae, such as Acrolepiopsis sapporensis, and Acrolepiopsis suzukiella, of Acrolepiopsis spp.; and others such as Plutella xylostella;
(p) Moths belonging to the family Pyralidae, such as Cadra cautella, Elasmopalpus lignosellus, Etiella zinckenella, and Galleria mellonella;
(q) Moths belonging to the family Sphingidae, such as Manduca quinquemaculata, and Manduca sexta, of Manduca spp.;
(r) Moths belonging to the family Stathmopodidae, such as Stathmopoda masinissa;
(s) Moths belonging to the family Tineidae, such as Tinea translucens;
(t) Moths belonging to the family Tortricidae, such as Adoxophyes honmai, and Adoxophyes orana, of Adoxophyes spp.; Archips breviplicanus, and Archips fuscocupreanus of Archips spp.; and others such as Choristoneura fumiferana, Cydia pomonella, Eupoecilia ambiguella, Grapholitha molesta, Homona magnanima, Leguminivora glycinivorella, Lobesia botrana, Matsumuraeses phaseoli, Pandemis heparana, and Sparganothis pilleriana;
(u) Moths belonging to the family Yponomeutidae, such as Argyresthia conjugella.
(2) Insect pests of the order Thysanoptera
(a) Insect pests belonging to the family Phlaeothripidae, such as Ponticulothrips diospyrosi;
(b) Insect pests belonging to the family Thripidae, such as Frankliniella intonsa, and Frankliniella occidentalis, of Frankliniella spp.; Thrips palmi, and Thrips tabaci, of Thrips spp.; and others such as Heliothrips haemorrhoidalis, and Scirtothrips dorsalis.
(3) Insect pests of the order Hemiptera
(A) The infraorder Archaeorrhyncha
(a) Insect pests belonging to the family Delphacidae, such as Laodelphax striatella, Nilaparvata lugens, Perkinsiella saccharicida, and Sogatella furcifera.
(B) The infraorder Clypeorrhyncha
(a) Insect pests belonging to the family Cicadellidae, such as Empoasca fabae, Empoasca nipponica, Empoasca onukii, and Empoasca sakaii, of Empoasca spp.; and others such as Arboridia apicalis, Balclutha saltuella, Epiacanthus stramineus, Macrosteles striifrons, and Nephotettix cinctinceps.
(C) The infraorder Heteroptera
(a) Insect pests belonging to the family Alydidae, such as Riptortus clavatus;
(b) Insect pests belonging to the family Coreidae, such as Cletus punctiger, and Leptocorisa chinensis;
(c) Insect pests belonging to the family Lygaeidae, such as Blissus leucopterus, Cavelerius saccharivorus, and Togo hemipterus;
(d) Insect pests belonging to the family Miridae, such as Halticus insularis, Lygus lineolaris, Psuedatomoscelis seriatus, Stenodema sibiricum, Stenotus rubrovittatus, and Trigonotylus caelestialium;
(e) Insect pests belonging to the family Pentatomidae, such as Nezara antennata, and Nezara viridula, of Nezara spp.; Eysarcoris aeneus, Eysarcoris lewisi, and Eysarcoris ventralis, of Eysarcoris spp.; and others such as Dolycoris baccarum, Eurydema rugosum, Glaucias subpunctatus, Halyomorpha halys, Piezodorus hybneri, Plautia crossota, and Scotinophora lurida;
(f) Insect pests belonging to the family Pyrrhocoridae, such as Dysdercus cingulatus;
(g) Insect pests belonging to the family Rhopalidae, such as Rhopalus msculatus;
(h) Insect pests belonging to the family Scutelleridae, such as Eurygaster integriceps);
(i) Insect pests belonging to the family Tingidae, such as Stephanitis nashi.
(D) The infraorder Sternorrhyncha
(a) Insect pests belonging to the family Adelgidae, such as Adelges laricis;
(b) Insect pests belonging to the family Aleyrodidae, such as Bemisia argentifolii, and Bemisia tabaci, of Bemisia spp.; and others such as Aleurocanthus spiniferus, Dialeurodes citri, and Trialeurodes vaporariorum;
(c) Insect pests belonging to the family Aphididae, such as Aphis craccivora, Aphis fabae, Aphis forbesi, Aphis gossypii, Aphis pomi, Aphis sambuci, and Aphis spiraecola, of Aphis spp.; Rhopalosiphum maidis, and Rhopalosiphum padi, of Rhopalosiphum spp.; Dysaphis plantaginea, and Dysaphis radicola, of Dysaphis spp.; Macrosiphum avenae, and Macrosiphum euphorbiae, of Macrosiphum spp.; Myzus cerasi, Myzus persicae, and Myzus varians, of Myzus spp.; and others such as Acyrthosiphon pisum, Aulacorthum solani, Brachycaudus helichrysi, Brevicoryne brassicae, Chaetosiphon fragaefolii, Hyalopterus pruni, Hyperomyzus lactucae, Lipaphis erysimi, Megoura viciae, Metopolophium dirhodum, Nasonovia ribis-nigri, Phorodon humuli, Schizaphis graminum, Sitobion avenae, and Toxoptera aurantii;
(d) Insect pests belonging to the family Coccidae, such as Ceroplastes ceriferus, and Ceroplastes rubens, of Ceroplastes spp.;
(e) Insect pests belonging to the family Diaspididae, such as Pseudaulacaspis pentagona, and Pseudaulacaspis prunicola, of Pseudaulacaspis spp.; Unaspis euonymi, and Unaspis yanonensis, of Unaspis spp.; and others such as Aonidiella aurantii, Comstockaspis perniciosa, Fiorinia theae, and Pseudaonidia paeoniae;
(f) Insect pests belonging to the family Margarodidae, such as Drosicha corpulenta, and Teerya purchasi;
(g) Insect pests belonging to the family Phylloxeridae, such as Viteus vitifolii;
(h) Insect pests belonging to the family Pseudococcidae, such as Planococcus citri, and Planococcus kuraunhiae, of Planococcus spp.; and others such as Phenacoccus solani, and Pseudococcus comstocki;
(i) Insect pests belonging to the family Psyllidae, such as Psylla mali, and Psylla pyrisuga, of Psylla spp.; and other such as Diaphorina citri.
(4) Insect pests of the infraorder Polyphaga
(a) Insect pests belonging to the family Anobiidae, such as Lasioderma serricorne;
(b) Insect pests belonging to the family Attelabidae, such as Byctiscus betulae, and Rhynchites heros;
(c) Insect pests belonging to the family Bostrichidae, such as Lyctus brunneus;
(d) Insect pests belonging to the family Brentidae, such as Cylas formicarius;
(e) Insect pests belonging to the family Buprestidae, such as Agrilus sinuatus;
(f) Insect pests belonging to the family Cerambycidae, such as Anoplophora malasiaca, Monochamus alternatus, Psacothea hilaris, and Xylotrechus pyrrhoderus;
(g) Insect pests belonging to the family Chrysomelidae, such as Bruchus pisorum, and Bruchus rufimanus, of Bruchus spp.; Diabrotica barberi, Diabrotica undecimpunctata, and Diabrotica virgifera, of Diabrotica spp.; Phyllotreta nemorum, and Phyllotreta striolata, of Phyllotreta spp.; and others such as Aulacophora femoralis, Callosobruchus chinensis, Cassida nebulosa, Chaetocnema concinna, Leptinotarsa decemlineata, Oulema oryzae, and Psylliodes angusticollis;
(h) Insect pests belonging to the family Coccinellidae, such as Epilachna varivestis, and Epilachna vigintioctopunctata, of Epilachna spp.;
(i) Insect pests belonging to the family Curculionidae, such as Anthonomus grandis, and Anthonomus pomorum, of Anthonomus spp.; Sitophilus granarius, and Sitophilus zeamais, of Sitophilus spp.; and others such as Echinoenemus squameus, Euscepes postfasciatus, Hylobius abietis, Hypera postica, Lissohoptrus oryzophilus, Otiorhynchus sulcatus, Sitona lineatus, and Sphenophorus venatus;
(j) Insect pests belonging to the family Elateridae, such as Melanotus fortnumi, and Melanotus tamsuyensis, of Melanotus spp.;
(k) Insect pests belonging to the family Nitidulidae, such as Epuraea domina;
(l) Insect pests belonging to the family Scarabaeidae, such as Anomala cuprea, and Anomala rufocuprea, of Anomala spp.; and others such as Cetonia aurata, Gametis jucunda, Heptophylla picea, Melolontha melolontha, and Popillia japonica;
(m) Insect pests belonging to the family Scolytidae, such as Ips typographus;
(n) Insect pests belonging to the family Staphylinidae, such as Paederus fuscipes;
(o) Insect pests belonging to the family Tenebrionidae, such as Tenebrio molitor, and Tribolium castaneum;
(p) Insect pests belonging to the family Trogossitidae, such as Tenebroides mauritanicus.
(5) Insect pests of the order Diptera
(A) The infraorder Brachycera
(a) Insect pests belonging to the family Agromyzidae, such as Liriomyza bryoniae, Liriomyza chinensis, Liriomyza sativae, and Liriomyza trifolii, of Liriomyza spp.; and others such as Chromatomyia horticola, and Agromyza oryzae;
(b) Insect pests belonging to the family Anthomyiidae, such as Delia platura, and Delia radicum, of Delia spp.; and others such as Pegomya cunicularia;
(c) Insect pests belonging to the family Drosophilidae, such as Drosophila melanogaster, and Drosophila suzukii, of Drosophila spp.;
(d) Insect pests belonging to the family Ephydridae, such as Hydrellia griseola;
(e) Insect pests belonging to the family Psilidae, such as Psila rosae;
(f) Insect pests belonging to the family Tephritidae, such as Bactrocera cucurbitae, and Bactrocera dorsalis, of Bactrocera spp.; Rhagoletis cerasi, and Rhagoletis pomonella, of Rhagoletis spp.; and others such as Ceratitis capitata, and Dacus oleae.
(B) The infraorder Nematocera
(a) Insect pests belonging to the family Cecidomyiidae, such as Asphondylia yushimai, Contarinia sorghicola, Mayetiola destructor, and Sitodiplosis mosellana.
(6) Insect pests of the order Orthoptera
(a) Insect pests belonging to the family Acrididae, such as Schistocerca americana, and Schistocerca gregaria, of Schistocerca spp.; and others such as Chortoicetes terminifera, Dociostaurus maroccanus, Locusta migratoria, Locustana pardalina, Nomadacris septemfasciata, and Oxya yezoensis;
(b) Insect pests belonging to the family Gryllidae, such as Acheta domestica, and Teleogryllus emma;
(c) Insect pests belonging to the family Gryllotalpidae, such as Gryllotalpa orientalis;
(d) Insect pests belonging to the family Tettigoniidae, such as Tachycines asynamorus.

### (7) Acarians (Acari)

(A) Acaridida of the order Astigmata
(a) Acarians belonging to the family Acaridae, such as Rhizoglyphus echinopus, and Rhizoglyphus robini, of Rhizoglyphus spp.; Tyrophagus neiswanderi, Tyrophagus perniciosus, Tyrophagus putrescentiae, and Tyrophagus similis, of Tyrophagus spp.; and others such as Acarus siro, Aleuroglyphus ovatus, and Mycetoglyphus fungivorus;
(B) Actinedida of the order Prostigmata
(a) Acarians belonging to the family Tetranychidae, such as Bryobia praetiosa, and Bryobia rubrioculus, of Bryobia spp.; Eotetranychus asiaticus, Eotetranychus boreus, Eotetranychus celtis, Eotetranychus geniculatus, Eotetranychus kankitus, Eotetranychus pruni, Eotetranychus shii, Eotetranychus smithi, Eotetranychus suginamensis, and Eotetranychus uncatus, of Eotetranychus spp.; Oligonychus hondoensis, Oligonychus ilicis, Oligonychus karamatus, Oligonychus mangiferus, Oligonychus orthius, Oligonychus perseae, Oligonychus pustulosus, Oligonychus shinkajii, and Oligonychus ununguis, of Oligonychus spp.; Panonychus citri, Panonychus mori, and Panonychus ulmi, of Panonychus spp.; Tetranychus cinnabarinus, Tetranychus evansi, Tetranychus kanzawai, Tetranychus ludeni, Tetranychus quercivorus, Tetranychus phaselus, Tetranychus urticae, and Tetranychus viennensis, of Tetranychus spp.; Aponychus corpuzae, and Aponychus firmianae, of Aponychus spp.; Sasanychus akitanus, and Sasanychus pusillus, of Sasanychus spp.; Shizotetranychus celarius, Shizotetranychus longus, Shizotetranychus miscanthi, Shizotetranychus recki, and Shizotetranychus schizopus, of Shizotetranychus spp.; and others such as Tetranychina harti, Tuckerella pavoniformis, and Yezonychus sapporensis;
(b) Acarians belonging to the family Tenuipalpidae, such as Brevipalpus lewisi, Brevipalpus obovatus, Brevipalpus phoenicis, Brevipalpus russulus, and Brevipalpus californicus, of Brevipalpus spp.; Tenuipalpus pacificus, and Tenuipalpus zhizhilashviliae, of Tenuipalpus spp.; and others such as Dolichotetranychus floridanus;
(c) Acarians belonging to the family Eriophyidae, such as Aceria diospyri, Aceria ficus, Aceria japonica, Aceria kuko, Aceria paradianthi, Aceria tiyingi, Aceria tulipae, and Aceria zoysiea, of Aceria spp.; Eriophyes chibaensis, and Eriophyes emarginatae, of Eriophyes spp.; Aculops lycopersici, and Aculops pelekassi, of Aculops spp.; Aculus fockeui, and Aculus schlechtendali, of Aculus spp.; and others such as Acaphylla theavagrans, Calacarus carinatus, Colomerus vitis, Calepitrimerus vitis, Epitrimerus pyri, Paraphytoptus kikus, Paracalacarus podocarpi, and Phyllocotruta citri;
(d) Acarians belonging to the family Transonemidae, such as Tarsonemus bilobatus, and Tarsonemus waitei, of Tarsonemus spp.; and others such as Phytonemus pallidus, and Polyphagotarsonemus latus;
(e) Acarians belonging to the family Penthaleidae, such as Penthaleus erythrocephalus, and Penthaleus major, of Penthaleus spp.

The pest control agent according to the present invention may be mixed or used with other active ingredients of fungicides, insecticides, acaricides, nematicides, or soil insect pest control agents, or plant regulatory agents, synergists, fertilizers, soil improvement agents, or animal feeds.

The combination of the compound according to the present invention with other active ingredients may exhibit synergistic effects on insecticidal, acaricidal, or nematicidal activity. The synergistic effects can be confirmed by a commonly used method using the Colby formula (Colby. S. R., Calculating Synergistic and Antagonistic Responses of Herbicide Combinations; Weeds 15, pages 20 to 22, 1967).

Specific examples of the insecticides, acaricides, nematicides, soil insect pest control agents, and anthelmintic agents which can be mixed or used with the pest control agent according to the present invention are shown below.

(1) Acetylcholinesterase inhibitors:
(a) Carbamate-based: alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, oxamyl, pirimicarb, propoxur, thiodicarb, thiofanox, triazamate, trimethacarb, XMC, xylylcarb, fenothiocarb, MIPC, MPMC, MTMC, aldoxycarb, allyxycarb, aminocarb, bufencarb, chloethocarb, metam sodium, promecarb;
(b) Organophosphate-based: acephate, azamethiphos, azinphos-ethyl, azinphos-methyl, cadusafos, chloroethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isocarbophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimphos, temephos, terbufos, tetrachlorvinphos, thiometon, triazophos, trichlorfon, vamidothion, bromophos-ethyl, BRP, carbophenothion, cyanofenphos, demeton-S methyl sulfone, dialifos, dichlofenthion, dioxabenzofos, etrimfos, fensulfothion, flupyrazofos, fonofos, formothion, phosmethylan, isazophos, iodofenphos, methacrifos, pirimiphos-ethyl, phosphocarb, propaphos, prothoate, sulprophos.
(2) GABAergic chloride ion channel antagonists: acetoprole, chlordene, endosulfan, ethiprole, fipronil, pyrafluoprole, pyriprole, camphlechlor, heptachlor, dienochlor.
(3) Sodium channel modulators: acrinathrin, d-cis-trans allethrin, d-trans allethrin, bifenthrin, bioallethrin, bioallethrin S-cyclopentyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R)-isomer], esfenvalerate, ethofenprox, fenpropathrin, fenvalerate, flucythrinate, flumethrin, tau-fluvalinate, halfenprox, imiprothrin, kadethrin, permethrin, phenothrin [(1R)-trans isomer], prallethrin, pyrethrum, resmethrin, silafluofen, tefluthrin, tetramethrin [(1R)-isomer], tralomethrin, transfluthrin, allethrin, pyrethrin, pyrethrin I, pyrethrin II, profluthrin, dimefluthrin, bioethanomethrin, biopermethrin, transpermethrin, fenfluthrin, fenpirithrin, flubrocythrinate, flufenprox, metofluthrin, protrifenbute, pyresmethrin, terallethrin.
(4) Nicotinic acetylcholine receptor agonist: acetamiprid, clothianidin, dinotefuran, imidacloprid, nitenpyram, nithiazine, thiacloprid, thiamethoxam, sulfoxaflor, nicotine, flupyradifurone, flupyrimin.
(5) Nicotinic acetylcholine receptor allosteric modulators: spinetoram, spinosad.
(6) Chloride channel activators: abamectin, emamectin benzoate, lepimectin, milbemectin, ivermectin, selamectin, doramectin, eprinomectin, moxidectin, milbemycin, milbemycin oxime, nemadectin.
(7) Juvenile hormone-like substances: hydroprene, kinoprene, methoprene, fenoxycarb, pyriproxyfen, diofenolan, epofenonane, triprene.
(8) Other non-specific inhibitors: methyl bromide, chloropicrin, sulfuryl fluoride, borax, tartar emetic.
(9) Homoptera selective feeding inhibitors: flonicamid, pymetrozine, pyrifluquinazon.
(10) Acarian growth inhibitors: clofentezine, diflovidazin, hexythiazox, etoxazole.
(11) Insectan midgut inner membrane disrupting agent derived from microorganisms: Bacillus thuringiensis subspecies Isuraerenshi, Bacillus sphaericus, Bacillus thuringiensis subsp. Aizawai, Bacillus thuringiensis subspecies Kurstaki, Bacillus thuringiensis subspecies Tenebrionis, Bt crop protein, Cry1 Ab, Cry1Ac, Cry1Fa, Cry1A.105, Cry2Ab, Vip3A, mCry3A, Cry3Ab, Cry3Bb, Cry34Ab1/Cry35Ab1.
(12) Mitochondrial ATP biosynthetic enzyme inhibitors: diafenthiuron, azocyclotin, cyhexatin, fenbutatin oxide, propargite, tetradifon.
(13) Oxidative phosphorylation uncouplers: chlorfenapyr, sulfluramid, DNOC, binapacryl, dinobuton, dinocap.
(14) Nicotinic acetylcholine receptor channel blockers: bensultap, cartap hydrochloride, nereistoxin, thiosultap-sodium, thiocyclam.
(15) Chitin synthesis inhibitors: bistrifluron, chlorfluazuron, diflubenzuron, flucycloxuron, flufenoxuron, hexaflumuron, lufenuron, novaluron, noviflumuron, teflubenzuron, triflumuron, buprofezin, fluazuron.
(16) Diptera molting disrupting agents: cyromazine.
(17) Molting hormone receptor agonists: chromafenozide, halofenozide, methoxyfenozide, tebufenozide.
(18) Octopamine receptor agonists: amitraz, demiditraz, chlordimeform.
(19) Mitochondrial electron transport system complex III inhibitors: acequinocyl, fluacrypyrim, hydramethylnon, bifenazate.
(20) Mitochondrial electron transport system complex I inhibitors: fenazaquin, fenpyroximate, pyrimidifen, pyridaben, tebufenpyrad, tolfenpyrad, rotenone.
(21) Voltage-dependent sodium channel blockers: indoxacarb, metaflumizone.
(22) Acetyl CoA carboxylase inhibitors: spirodiclofen, spiromesifen, spirotetramat, spiropidion.
(23) Mitochondrial electron transport system complex IV inhibitors: aluminum phosphide, calcium phosphide, phosphine, zinc phosphide, cyanide.
(24) Mitochondrial electron transport system complex II inhibitors: cyenopyrafen, cyflumetofen, pyflubumide.
(25) Ryanodine receptor modulators: chlorantraniliprole, cyantraniliprole, flubendiamide, cyclaniliprole, tetraniliprole.
(26) Mixed function oxidase inhibitor compounds: piperonyl butoxide.
(27) Latrophilin receptor agonists: depsipeptide, cyclic depsipeptide, 24-membered cyclic depsipeptide, emodepside.
(28) Other agents (mechanisms of which are unknown): acynonapyr, azadirachtin, benzoximate, bromopropylate, chinomethionat, cryolite, dicofol, pyridalyl, benclothiaz, sulfur, amidoflumet, 1,3-dichloropropene, DCIP, phenisobromolate, benzomate, metaldehyde, chlorobenzilate, clothiazoben, dicyclanil, fenoxacrim, fentrifanil, flubenzimin, fluphenazine, gossyplure, japonilure, metoxadiazone, petroleum, sodium oleate, tetrasul, triarathene, afidopyropen, flometoquin, flufiprole, fluensulfone, meperfluthrin, tetramethylfluthrin, tralopyril, methylneodecanamide, fluralaner, afoxolaner, fluxametamide, 5-[5-(3,5-dichlorophenyl)-5-trifluoromethyl-4,5-dihydroisoxazol-3-yl]-2-(1H-1,2,4-triazol-1-yl) benzonitrile (CAS: 943137-49-3), broflanilide, triflumezopyrim, dicloromezotiaz, oxazosulfyl, other meta-diamides, tyclopyrazoflor.
(29) Anthelmintic agents:
(a) Benzimidazole-based: fenbendazole, albendazole, triclabendazole, oxibendazole, mebendazole, oxfendazole, parbendazole, flubendazole, febantel, netobimin, thiophanate, thiabendazole, cambendazole;
(b) Salicylanilide-based: closantel, oxyclozanide, rafoxanide, niclosamide;
(c) Substituted phenol-based: nitroxinil, nitroscanate;
(d) Pyrimidine-based: pyrantel, morantel;
(e) Imidazothiazole-based: levamisole, tetramisole;
(f) Tetrahydropyrimidine-based: praziquantel, epsiprantel;
(g) Other anthelmintic agents: cyclodiene, ryania, clorsulon, metronidazole, demiditraz, piperazine, diethylcarbamazine, dichlorophene, monepantel, tribendimidine, amidantel, thiacetarsamide, melarsomine, arsenamide.

Specific examples of fungicides which may be mixed or used with the pest control agent according to the present invention are shown below.

(A) Nucleic acid biosynthesis inhibitors
(a) RNA polymerase I inhibitors: benalaxyl, benalaxyl-M, furalaxyl, metalaxyl, metalaxyl-M, oxadixyl, clozylacon, ofurace.
(b) Adenosine deaminase inhibitors: bupirimate, dimethirimol, ethirimol.
(c) DNA/RNA synthesis inhibitors: hymexazol, octhilinone.
(d) DNA topoisomerase II inhibitors: oxolinic acid.
(2) Karyokinesis inhibitors and cell division inhibitors
(a) β-tubulin polymerization inhibitors: benomyl, carbendazim, chlorfenazole, fuberidazole, thiabendazole, thiophanate, thiophanate-methyl, diethofencarb, zoxamide, ethaboxam;
(b) Cell division inhibitors: pencycuron
(c) Delocalization inhibitor of spectrin-like protein: fluopicolide.
(3) Respiration inhibitor:
(a) Complex I NADH oxidation-reduction inhibitor: diflumetorim; tolfenpyrad;
(b) Complex II succinic acid dehydrogenase inhibitor: benodanil, flutolanil, mepronil, isofetamid, fluopyram, fenfuram, furmecyclox, carboxin, oxycarboxin, thifluzamide, benzovindiflupyr, bixafen, fluxapyroxad, furametpyr, isopyrazam, penflufen, penthiopyrad, sedaxane, boscalid, pyrapropoyne;
(c) Complex III ubiquinol oxidase Qo inhibitor: azoxystrobin, coumoxystrobin, coumethoxystrobin, enoxastrobin, flufenoxystrobin, picoxystrobin, pyraoxystrobin, pyraclostrobin, pyrametostrobin, triclopyricarb, kresoxim-methyl, trifloxystrobin, dimoxystrobin, fenaminstrobin, metominostrobin, orysastrobin, famoxadone, fluoxastrobin, fenamidone; pyribencarb;
(d) Complex III ubiquinol reductase Qi inhibitor: cyazofamid, amisulbrom;
(e) Oxidative phosphorylation uncoupling agent: binapacryl, meptyldinocap, dinocap, fluazinam, ferimzone;
(f) Oxidative phosphorylation inhibitor (ATP synthase inhibitor): fenthin acetate, fentin chloride, fentin hydroxide;
(g) ATP production inhibitor: silthiofam;
(h) Complex III cytochrome bcl (ubiquinone reductase) Qx (unknown) inhibitor: ametoctradin;
(4) Amino acid and protein synthesis inhibitor
(a) Methionine biosynthesis inhibitor: andoprim, cyprodinil, mepanipyrim, pyrimethanil;
(b) Protein synthesis inhibitor: blasticidin-S, kasugamycin, kasugamycin hydrochloride, streptomycin, oxytetracycline;
(5) Signal transfer inhibitor:
(a) Signal transfer inhibitor: quinoxyfen, proquinazid;
(b) MAP/histidine kinase inhibitor in osmotic pressure signal transfer: fenpiconil, fludioxonil, chlozolimate, iprodione, procymidone, vinclozolin;
(6) Lipid and cell membrane synthesis inhibitor:
(a) Phospholipid biosynthesis and methyltransferase inhibitor: edifenphos, iprobenfos, pyrazophos, isoprothiolane;
(b) Lipid peroxide agent: biphenyl, chloroneb, dichloran, quintozene, tecnazene, tolclofos-methyl; etridiazole;
(c) Agents affecting cell membrane: iodocarb, propamocarb, propamocarb hydrochloride, propamocarb-fosetylate, prothiocarb;
(d) Microorganisms disturbing cell membrane of pathogenic bacteria: Bacillus subtilis, Bacillus subtilis strain QST713, Bacillus subtilis strain FZB24, Bacillus subtilis strain MB1600, Bacillus subtilis strain D747;
(e) Agents disturbing cell membrane: melaleuca alternifolia (tea tree) extract.
(7) Cell membrane sterol biosynthesis inhibitor:
(a) C14 position demethylation inhibitor in sterol biosynthesis: triforine; pyrifenox, pyrisoxazole; fenarimol, flurprimidol, nuarimol, imazalil, imazalil-sulphate, oxpoconazole, pefurazoate, prochloraz, triflumizole, viniconazole, azaconazole, bitertanol, bromconazole, cyproconazole, diclobutrazol, difenoconazole, diniconazole, diniconazole-M, epoxyconazole, etaconazole, fenbuconazole, fluquinconazole, flusilazole, flutriafol, furconazole, furconazole-cis, hexaconazole, imibenconazole, ipuconazole, metconazole, myclobutanil, penconazole, propiconazole, fluquinconazole, simeconazole, tebuconazole, tetraconazole, triadimefon, triadimenol, triticonazole, prothioconazole, voriconazole;
(b) Δ14 reductase and Δ8→Δ7-isomerase inhibitor in sterol biosynthesis: aldimorph, dodemorph, dodemorph-acetate, fenpropimorph, tridemorph, fenpropidine, piperalin, spiroxamine;
(c) 3-Keto reductase inhibitor in C4 position demethylation in sterol biosynthesis system: fenhexamid, fenpyrazamine;
(d) Squalene epoxidase inhibitor in sterol biosynthesis system: pyributicarb, naftifen, terbinafine;
(8) Cell wall synthesis inhibitor
(a) Trehalase inhibitor: validamycin;
(b) Chitin synthetase inhibitor: polyoxin, polyoxorim;
(c) Cellulose synthetase inhibitor: dimethomorph, flumorph, pyrimorph, benthiavalicarb, iprovalicarb, tolprocarb, valifenalate, mandipropamide;
(9) Melanin biosynthesis inhibitor
(a) Reductase inhibitor in melanin biosynthesis: fthalide, pyroquilon, tricyclazole;
(b) Anhydrase inhibitor in melanin biosynthesis: carpropamid, diclocymet, fenoxanil;
(10) Resistance-inducing agent of host plant:
(a) Agents affecting salicylic acid synthetic pathway: acibenzolar-s-methyl;
(b) Others: probenazole, tiadinil, isotianil, laminarin, extract liquid of Reynoutria sachalinensis;
(11) Agents of which the activity is unknown: cymoxanil, fosetyl-aluminum, phosphoric acid (phosphate), tecloftalam, triazoxide, flusulfamide, diclomezine, methasulfocarb, cyflufenamid, metrafenone, pyriofenone, dodine, dodine free base, flutianil;
(12) Agent having multiple activities: copper (copper salt), bordeaux mixture, copper hydroxide, copper naphthalate, copper oxide, oxychloride copper, copper sulfate, sulfur, sulfur product, calcium polysulfide, ferbam, mancozeb, maneb, mancopper, metiram, polycarbamate, propineb, thiram, zineb, ziram, captan, captafol, folpet, chlorothalonil, dichlofluanid, tolylfluanid, guazatine, iminoctadine triacetate, iminoctadine trialbesilate, anilazine, dithianon, chinomethionat, fluoroimide;
(13) Other agents: DBEDC, fluor folpet, guazatine acetate, bis (8-quinolinolato) copper (II), propamidine, chloropicrin, cyprofuram, agrobacterium, bethoxazin, diphenylamine, methyl isothiocyanate (MITC), mildew-mycin, capsaicin, curfraneb, cyprosulfamide, dazomet, debacarb, dichlorophen, difenzoquat, difenzoquat⋅ methyl sulfonate, flumetover, fosetyl-calcium, fosetyl-sodium, irmamycin, natamycin, nitrothal isopropyl, oxamocarb, puropamocin sodium, pyrrolnitrin, tebufloquin, tolnifanide, zarilamide, algophase, amicarthiazol, oxathiapiprolin, metiram zinc, benthiazole, trichlaamide, uniconazole, mildew-mycin, oxyfenthiin, picarbutrazox.

Specific examples of plant regulatory agents which can be mixed or used with the pest control agent according to the present invention are shown below.

1-Methylcyclopropene, 2,3,5-triiodobenzoic acid, IAA, IBA, MCPA, MCPB, 4-CPA, 5-aminolevulinic acid hydrochloride, 6-benzylaminopurine, abscisic acid, aviglycine hydrochloride, ancymidol, butralin, calcium carbonate, calcium chloride, calcium formate, calcium peroxide, lime sulfur, calcium sulfate, chlormequat chloride, chlorpropham, choline chloride, cloprop, cyanamide, cyclanilide, daminozide, decyl alcohol, dichlorprop, dikegulac, dimethipin, diquat, ethephon, ethychlozate, flumetralin, flurprimidol, forchlorfenuron, gibberellin A, gibberellin A3, hymexazol, inabenfide, isoprothiolane, kinetin, maleic acid hydrazide, mefluidide, mepiquat chloride, oxidation type glutathione, paclobutrazol, pendimethalin, prohexadione calcium, prohydrojasmon, pyraflufen-ethyl, sintofen, sodium 1-naphthalene acetate, sodium cyanate, streptomycin, thidiazuron, triapenthenol, tribufos, trinexapac-ethyl, uniconazole P, and 1-nathtylacetamide.

(Ectoparasite control Agent)

An ectoparasite control agent according to the present invention contains at least one selected from the pyridinium salts according to the present invention, as an active ingredient thereof. Although the amount of the compound according to the present invention contained in the ectoparasite control agent according to the present invention is not particularly limited within the range in which ectoparasite control effects are exhibited, the amount of the compound according to the present invention relative to 100 parts by mass of the control agent according to the present invention is generally preferably 0.01 parts by mass to 95 parts by mass.

Examples of host animals to be treated with the ectoparasite control agent according to the present invention include warm-blooded animals such as: pet animals such as dogs or cats; pet birds; farm animals such as cattle, horses, pigs, and sheep; and domestic fowl. Additional examples thereof include honey-bees, stag beetles, and unicorn beetles.

The ectoparasite control agent according to the present invention may be applied by a known veterinary method (topical, oral, parenteral or subcutaneous administration). Examples of the method include: a method in which a tablet, capsule or feed mixed with the ectoparasite control agent is orally administered to the animals; a method in which an immersion liquid, suppository or injection (intramuscular, subcutaneous, intravenous, intraabdominal or the like) is administered to the animals; a method in which an oil-based or aqueous liquid preparation is topically administered by conducting spraying, pouring on, spotting on or the like; and a method in which the ectoparasite control agent is topically administered in an appropriate form to animals by attaching a collar, an ear tag or the like made by molding a mixture obtained by kneading the ectoparasite control agent into a resin.

Ectoparasites parasitize host animals, especially parasitize in or on the body of warm-blooded animals. More specifically, ectoparasites parasitize the back, armpit, underbelly, inner thigh or the like of host animals and obtain nutritional sources such as blood or dandruff from animals to live. Examples of ectoparasites include acarians, lice, fleas, mosquitoes, stable flies, and flesh flies. Specific examples of ectoparasites to be controlled by the ectoparasite control agent according to the present invention are shown below.

### (1) Acarians (Acari)

Acarians belonging to the family Dermanyssidae, acarians belonging to the family Macronyssidae, acarians belonging to the family Laelapidae, acarians belonging to the family Varroidae, acarians belonging to the family Argasidae, acarians belonging to the family Ixodidae, acarians belonging to the family Psoroptidae, acarians belonging to the family Sarcoptidae, acarians belonging to the family Knemidokoptidae, acarians belonging to the family Demodixidae, acarians belonging to the family Trombiculidae, and insect-parasitic acari such as Coleopterophagus berlesei.

### (2) Order Phthiraptera

Lice belonging to the family Haematopinidae, lice belonging to the family Linognathidae, biting lice belonging to the family Menoponidae, biting lice belonging to the family Philopteridae, and biting lice belonging to the family Trichodectidae.

### (3) Order Siphonaptera

Fleas belonging to the family Pulicidae, such as Ctenocephalides canis and Ctenocephalides felis of Ctenocephalides spp.; fleas belonging to the family Tungidae, fleas belonging to the family Ceratophyllidae, and fleas belonging to the family Leptopsyllidae.

### (4) Order Hemiptera.

(5) Insect pests of the order Diptera

Mosquitoes belonging to the family Culicidae, black flies belonging to the family Simuliidae, punkie belonging to the family Ceratopogonidae, horseflies belonging to the family Tabanidae, flies belonging to the family Muscidae, tsetse flies belonging to the family Glossinidae, flesh flies belonging to the family Sarcophagidae, flies belonging to the family Hippoboscidae, flies belonging to the family Calliphoridae, and flies belonging to the family Oestridae.

### (Endoparasite Control Agent or Expellant)

An endoparasite control agent or expellant according to the present invention contains at least one selected from the pyridinium salts according to the present invention, as an active ingredient thereof. Although the amount of the compound according to the present invention contained in the endoparasite control agent or expellant according to the present invention is not particularly limited within the range in which endoparasite control effects are exhibited, the amount of the compound according to the present invention relative to 100 parts by mass of the endoparasite control agent or expellant according to the present invention is generally preferably 0.01 parts by mass to 95 parts by mass.

Parasites to be controlled or eradicated by the endoparasite control agent or expellant according to the present invention parasitize in host animals, particularly in warm-blooded animals or fish (endoparasite). Examples of the host animals on which the endoparasite control agent or expellant according to the present invention is effective include: warm-blooded animals such as humans, domestic mammals (such as cows, horses, pigs, sheep, and goats), laboratory animals (such as mice, rats, and jirds), pet animals (such as hamsters, guinea pigs, dogs, cats, horses, squirrels, rabbits, and ferrets), mammals in nature or zoos (such as monkeys, foxes, deer, and buffalos), poultry (such as turkeys, ducks, chickens, quail, and geese), and pet birds (such as pigeons, parrots, magpies, java sparrows, parakeets, finches, and canaries); and fish such as salmon, trout, and koi carp. It is possible to prevent or treat parasitic diseases mediated by parasites by controlling or expelling the parasites.

Examples of the parasites to be controlled or eradicated include the following.

(1) Nematodes of the order Dioctophymatida
(a) Kidney worms belonging to the family Dioctophymatidae, such as Dioctophyma renale of Dioctophyma spp.; and
(b) kidney worms belonging to the family Soboliphymatidae, such as Soboliphyme abei and Soboliphyme baturini of Soboliphyme spp.
(2) Nematodes of the order Trichocephalida
(a) Trichinae belonging to the family Trichinellidae, such as Trichinella spiralis of Trichinella spp.; and
(b) whipworms belonging to the family Trichuridae, such as Capillaria annulata, Capillaria contorta, Capillaria hepatica, Capillaria perforans, Capillaria plica, and Capillaria suis, of Capillaria spp.; and Trichuris vulpis, Trichuris discolor, Trichuris ovis, Trichuris skrjabini, and Trichuris suis, of Trichuris spp.
(3) Nematodes of the order Rhabditida

Strongyloides stercoralis belonging to the family Strongyloididae, such as Strongyloides papillosus, Strongyloides planiceps, Strongyloides ransomi, Strongyloides suis, Strongyloides stercoralis, Strongyloides tumefaciens, and Strongyloides ratti, of Strongyloides spp.

(4) Nematodes of the order Strongylida

Ucinarias belonging to the family Ancylostomatidae, such as Ancylostoma braziliense, Ancylostoma caninum, Ancylostoma duodenale, and Ancylostoma tubaeforme, of Ancylostoma spp.; Uncinaria stenocephala of Uncinaria spp.; and Bunostomum phlebotomum, and Bunostomum trigonocephalum, of Bunostomum spp.

(5) Nematodes of the order Strongylida
(a) Nematodes belonging to the family Angiostrongylidae, such as Aelurostrongylus abstrusus of Aelurostrongylus spp.; and Angiostrongylus vasorum, and Angiostrongylus cantonesis, of Angiostrongylus spp.;
(b) nematodes belonging to the family Crenosomatidae, such as Crenosoma aerophila, and Crenosoma vulpis, of Crenosoma spp.;
(c) nematodes belonging to the family Filaroididae, such as Filaroides hirthi, and Filaroides osleri, of Filaroides spp.;
(d) metastrongyles belonging to the family Metastrongylidae, such as Metastrongylus apri, Metastrongylus asymmetricus, Metastrongylus pudendotectus, and Metastrongylus salmi, of Metastrongylus spp.; and
(e) gapeworms belonging to the family Syngamidae, such as Cyathostoma bronchialis of Cyathostoma spp.; and Syngamus skrjabinomorpha, and Syngamus trachea, of Syngamus spp.
(6) Nematodes of the order Strongylida
(a) Nematodes belonging to the family Molineidae, such as Nematodirus filicollis, and Nematodirus spathiger, of Nematodirus spp.;
(b) nematodes belonging to the family Dictyocaulidae, such as Dictyocaulus filaria, and Dictyocaulus viviparus, of Dictyocaulus spp.;
(c) nematodes belonging to the family Haemonchidae, such as Haemonchus contortus of Haemonchus spp.; and Mecistocirrus digitatus of Mecistocirrus spp.;
(d) nematodes belonging to the family Haemonchidae, such as Ostertagia ostertagi of Ostertagia spp.;
(e) nematodes belonging to the family Heligmonellidae, such as Nippostrongylus braziliensis of Nippostrongylus spp.; and
(f) nematodes belonging to the family Trichostrongylidae, such as Trichostrongylus axei, Trichostrongylus colubriformis, and Trichostrongylus tenuis, of Trichostrongylus spp.; Hyostrongylus rubidus of Hyostrongylus spp.; and Obeliscoides cuniculi of Obeliscoides spp.
(7) Nematodes of the order Strongylida
(a) Nematodes belonging to the family Chabertiidae, such as Chabertia ovina of Chabertia spp.; and Oesophagostomum brevicaudatum (pig), Oesophagostomum columbianum, Oesophagostomum dentatum, Oesophagostomum georgianum (pig), Oesophagostomum maplestonei, Oesophagostomum quadrispinulatum (pig), Oesophagostomum radiatum, Oesophagostomum venulosum, and Oesophagostomum watanabei (hog), of Oesophagostomum spp.;
(b) nematodes belonging to the family Stephanuridae, such as Stephanurus dentatus of Stephanurus spp.; and
(c) nematodes belonging to the family Strongylidae, such as Strongylus asini, Strongylus edentatus, Strongylus equinus, and Strongylus vulgaris, of Strongylus spp.
(8) Nematodes of the order Oxyurida

Nematodes belonging to the family Oxyuridae, such as Enterobius anthropopitheci, and Enterobius vermicularis, of Enterobius spp.; Oxyuris equi of Oxyuris spp.; and Passalurus ambiguus of Passalurus spp.

(9) Nematodes of the order Ascaridida
(a) Nematodes belonging to the family Ascaridiidae, such as Ascaridia galli of Ascaridia spp.;
(b) nematodes belonging to the family Heterakidae, such as Heterakis beramporia, Heterakis brevispiculum, Heterakis gallinarum, Heterakis pusilla, and Heterakis putaustralis, of Heterakis spp.;
(c) nematodes belonging to the family Anisakidae, such as Anisakis simplex of Anisakis spp.;
(d) nematodes belonging to the family Ascarididae, such as Ascaris lumbricoides, and Ascaris suum, of Ascaris spp.; and Parascaris equorum of Parascaris spp.; and
(e) nematodes belonging to the family Toxocaridae, such as Toxocara canis, Toxocara leonina, Toxocara suum, Toxocara vitulorum, and Toxocara cati, of Toxocara spp.
(10) Nematodes of the order Spirurida
(a) Nematodes belonging to the family Onchocercidae, such as Brugia malayi, Brugia pahangi, and Brugia patei, of Brugia spp.; Dipetalonema reconditum of Dipetalonema spp.; Dirofilaria immitis of Dirofilaria spp.; Filaria oculi of Filaria spp.; and Onchocerca cervicalis, Onchocerca gibsoni, and Onchocerca gutturosa, of Onchocerca spp.;
(b) nematodes belonging to the family Setariidae, such as Setaria digitata, Setaria equina, Setaria labiatopapillosa, and Setaria marshalli, of Setaria spp.; and Wuchereria bancrofti of Wuchereria spp.; and
(c) nematodes belonging to the family Filariidae, such as Parafilaria multipapillosa of Parafilaria spp.; and Stephanofilaria assamensis, Stephanofilaria dedoesi, Stephanofilaria kaeli, Stephanofilaria okinawaensis, and Stephanofilaria stilesi of Stephanofilaria spp.

### (11) Nematodes of the Order Spirurida

(a) Nematodes belonging to the family Gnathostomatidae, such as Gnathostoma doloresi, and Gnathostoma spinigerum, of Gnathostoma spp.;
(b) nematodes belonging to the family Habronematidae, such as Habronema majus, Habronema microstoma, and Habronema muscae, of Habronema spp.; and Draschia megastoma of Draschia spp.;
(c) nematodes belonging to the family Physalopteridae, such as Physaloptera canis, Physaloptera cesticillata, Physaloptera erdocyona, Physaloptera felidis, Physaloptera gemina, Physaloptera papilloradiata, Physaloptera praeputialis, Physaloptera pseudopraerutialis, Physaloptera rara, Physaloptera sibirica, and Physaloptera vulpineus, of Physaloptera spp.;
(d) nematodes belonging to the family Gongylonematidae, such as Gongylonema pulchrum of Gongylonema spp.;
(e) nematodes belonging to the family Spirocercidae, such as Ascarops strongylina of Ascarops spp.; and
(f) nematodes belonging to the family Thelaziidae, such as Thelazia callipaeda, Thelazia gulosa, Thelazia lacrymalis, Thelazia rhodesi, and Thelazia skrjabini, of Thelazia spp.

### (Control Agent Against Other Pests)

In addition, the compound according to the present invention exhibits an excellent effect of controlling insect pests that have a sting or venom that can harm humans or livestock, insect pests carrying various pathogens/pathogenic bacteria, or insect pests that impart a discomforting sensation to humans (such as toxic insect pests, sanitary insect pests, or unpleasant insect pests).

Specific examples thereof are shown below.

### (1) Insect Pests of the Order Hymenoptera

Sawflies belonging to the family Argidae, wasps belonging to the family Cynipidae, sawflies belonging to the family Diprionidae, ants belonging to the family Formicidae, wasps belonging to the family Mutillidae, and wasps belonging to the family Vespidae.

### (2) Other Insect Pests

Blattodea, termite, araneae, cetipede, millipede, crustacea and Cimex lectularius.

### EXAMPLES

### (Preparation Formulation)

Although some preparation formulations of the pest control agent, insecticide, acaricide, ectoparasite control agent, endoparasite control agent or expellant according to the present invention are shown below, additives and addition amounts thereof are not limited to these examples, and may be varied in a wide range. In the preparation formulation, the term “part” indicates “parts by mass” and the term “%” indicates “% by mass”.

The agricultural, horticultural or paddy preparation formulations are shown below.

### Formulation Example 1: Wettable Powders

40 parts of a compound according to the present invention, 53 parts of diatomaceous earth, 4 parts of higher alcohol sulfuric acid ester, and 3 parts of alkyl naphthalene sulfonate are mixed uniformly, and then finely pulverized to obtain wettable powders containing 40% of the active ingredient.

### Formulation Example 2: Emulsion

30 parts of a compound according to the present invention, 33 parts of xylene, 30 parts of dimethylformamide, and 7 parts of polyoxyethylene alkyl allyl ether are mixed and dissolved to obtain an emulsion containing 30% of the active ingredient.

### Formulation Example 3: Granules

5 parts of a compound according to the present invention, 40 parts of talc, 38 parts of clay, 10 parts of bentonite, and 7 parts of sodium alkyl sulfate are mixed uniformly, and then finely pulverized, followed by conducting granulation to obtain a particle diameter of 0.5 to 1.0 mm, and thus granules containing 5% of the active ingredient are obtained.

### Formulation Example 4: Granules

5 parts of a compound according to the present invention, 73 parts of clay, 20 parts of bentonite, 1 part of sodium dioctyl sulfosuccinate, and 1 part of potassium phosphate are mixed well and then pulverized, followed by adding water thereto, and then kneading the mixture. Then, granulation and drying are conducted to obtain granules containing 5% of the active ingredient.

### Formulation Example 5: Suspension

10 parts of a compound according to the present invention, 4 parts of polyoxyethylene alkyl allyl ether, 2 parts of sodium polycarboxylate, 10 parts of glycerin, 0.2 parts of xanthan gum, and 73.8 parts of water are mixed, and then wet-pulverized until the particle size becomes 3 m or less to obtain a suspension containing 10% of the active ingredient.

Preparation formulations of ectoparasite control agents, endoparasite control agents or expellants are shown below.

### Formulation Example 6: Granules

5 parts of a compound according to the present invention is dissolved in an organic solvent to obtain a solution. The solution is sprayed on a mixture of 94 parts of kaolin and 1 part of white carbon, followed by evaporating the solvent under reduced pressure to obtain granules. The granules may be mixed with animal feed to be used.

### Formulation Example 7: Injection Agent

0.1 to 1 part of a compound according to the present invention and 99 to 99.9 parts of peanut oil are mixed uniformly, and then filter-sterilized using a sterilizing filter.

### Formulation Example 8: Pour-on Agent

5 parts of a compound according to the present invention, 10 parts of a myristic acid ester and 85 parts of isopropanol are mixed uniformly to obtain a pour-on agent.

### Formulation Example 9: Spot-on Agent

10 to 15 parts of a compound according to the present invention, 10 parts of a palmitic acid ester and 75 to 80 parts of isopropanol are mixed uniformly to obtain a spot-on agent.

### Formulation Example 10: Spray Agent

1 part of a compound according to the present invention, 10 parts of propylene glycol and 89 parts of isopropanol are mixed uniformly to obtain a spray agent.

The present invention is explained further specifically by showing synthesis examples below. The present invention is not limited to the following examples.

### Example 1

### Synthesis of pyridin-1-ium-1-yl(4-((4-(trifluoromethyl)phenyl)ethynyl)benzoyl) amide

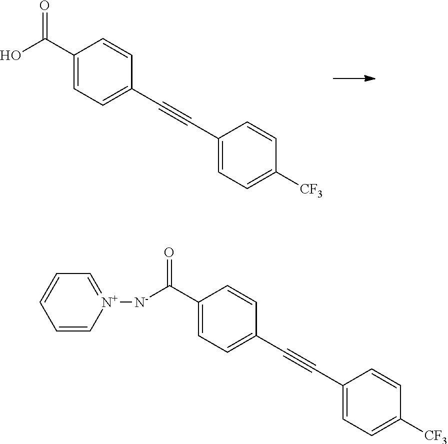 [SMILES: O=C(O)c1ccc(C#Cc2ccc(C(F)(F)F)cc2)cc1 ; O=C([N-][n+]1ccccc1)c1ccc(C#Cc2ccc(C(F)(F)F)cc2)cc1]

4-((4-(Trifluoromethyl)phenyl)ethynyl)benzoic acid (0.06 g) was dissolved in thionyl chloride (2 ml), and one drop of pyridine was added thereto, followed by stirring the mixture at room temperature for 3 hours. The resultant liquid was concentrated under reduced pressure. Chloroform (8 ml), 1-aminopyridinium iodide (0.05 g) and triethylamine (0.06 g) were added to the resultant concentrate, and then the mixture was stirred at room temperature for 1 hour. Water was added to the resultant, and the mixture was subjected to extraction with chloroform. An organic layer was dried with anhydrous magnesium sulfate and filtered, and then the solvent was distilled off. The resultant residue was washed by adding diethyl ether thereto, and thus 0.06 g of the titled compound was obtained at a yield of 75%.

### Example 2

### Synthesis of (E)-Pyridin-1-ium-1-yl(4-(4-(trifluoromethyl)styryl)benzoyl)amide

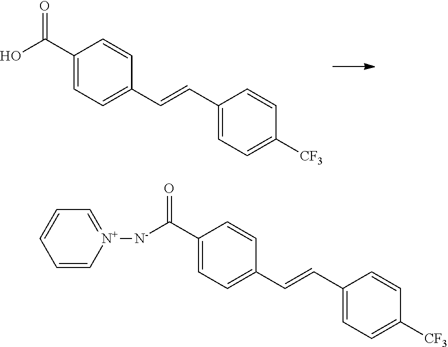 [SMILES: O=C(O)c1ccc(/C=C/c2ccc(C(F)(F)F)cc2)cc1 ; O=C([N-][n+]1ccccc1)c1ccc(/C=C/c2ccc(C(F)(F)F)cc2)cc1]

(E)-4-(4-(trifluoromethyl)styryl)benzoic acid (0.43 g) was dissolved in toluene (20 ml), and then thionyl chloride (0.54 g) and one drop of pyridine were added thereto, followed by stirring the mixture while heating the mixture to reflux for 1 hour. The resultant was concentrated under reduced pressure. Chloroform (20 ml), 1-aminopyridinium iodide (0.33 g) and triethylamine (0.45 g) were added to the resultant concentrate, and then stirred at room temperature for 30 minutes. Water was added to the resultant, and the mixture was subjected to extraction with chloroform. An organic layer was dried with anhydrous magnesium sulfate and filtered, and then the solvent was distilled off. The resultant residue was washed by adding diethyl ether and acetone thereto, and thus 0.25 g of the titled compound was obtained at a yield of 45%.

### Example 3

### Synthesis of (4-(2-(4-chlorophenyl)cyclopropyl)benzoyl)(pyridin-1-ium-1-yl) amide

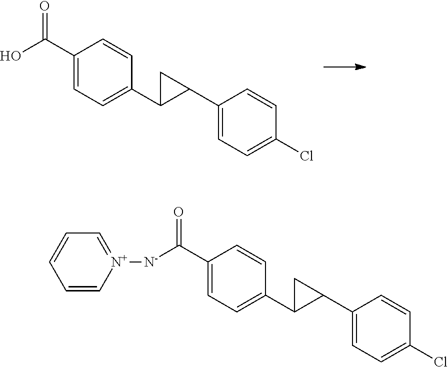 [SMILES: O=C(O)c1ccc(C2CC2c2ccc(Cl)cc2)cc1 ; O=C([N-][n+]1ccccc1)c1ccc(C2CC2c2ccc(Cl)cc2)cc1]

(4-(2-(4-Chlorophenyl)cyclopropyl)benzoic acid (0.17 g) was dissolved in dichloromethane (5 ml), and then oxalyl chloride (0.24 g) and one drop of DMF were added thereto, followed by stirring the mixture at room temperature for two hours. The resultant was concentrated under reduced pressure. Dichloromethane (8 ml), 1-aminopyridinium iodide (0.13 g) and triethylamine (0.38 g) were added to the resultant concentrate, followed by stirring the mixture at room temperature for one hour. Water was added to the resultant, and then the mixture was subjected to extraction with chloroform. An organic layer was dried with anhydrous magnesium sulfate and filtered, and then the solvent was distilled off. A mixture solvent composed of dichloromethane and methanol was added to the resultant residue to allow recrystallization to proceed, and thus 0.17 g of the titled compound was obtained at a yield of 90%.

### Example 4

### Synthesis of 1-(4-(4-(trifluoromethoxy)benzoyl)benzamide)pyridinium intramolecular salt

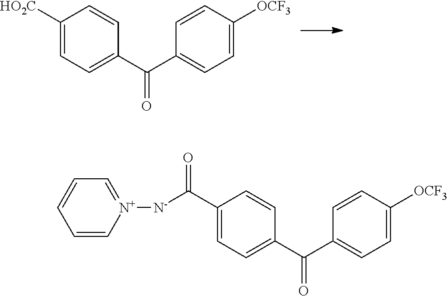 [SMILES: O=C(O)c1ccc(C(=O)c2ccc(OC(F)(F)F)cc2)cc1 ; O=C([N-][n+]1ccccc1)c1ccc(C(=O)c2ccc(OC(F)(F)F)cc2)cc1]

Thionyl chloride (5 ml) and pyridine (three drops) were added to 4-(4-(trifluoromethoxy)benzoyl)benzoic acid (0.87 g), and then refluxed for one hour. The resultant liquid was left to cool. Then, the solvent was distilled off. Chloroform (20 ml), N-aminopyridinium iodide (0.62 g) and triethylamine (0.85 ml) were added to the resultant residue, and then the mixture was stirred at room temperature for 17 hours. The resultant was put in an appropriate amount of water, and then subjected to extraction with chloroform. An organic layer was dried with anhydrous magnesium sulfate, and then the solvent was distilled off. The resultant solid was washed with diethyl ether to obtain the desired compound (0.6 g).

The 4-(4-(trifluoromethoxy)benzoyl)benzoic acid was prepared by the method described in WO2002092552.

### Example 5

### Synthesis of 1-(4-(3,3,3-trifluoro-1-propin-1-yl)benzamide)pyridinium intramolecular salt

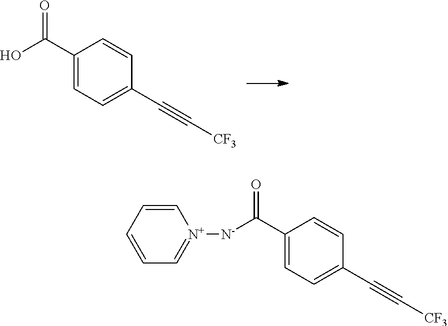 [SMILES: O=C(O)c1ccc(C#CC(F)(F)F)cc1 ; O=C([N-][n+]1ccccc1)c1ccc(C#CC(F)(F)F)cc1]

Thionyl chloride (5 ml) and pyridine (three drops) were added to 4-(3,3,3-trifluoro-1-propin-1-yl)benzoic acid (0.55 g), and then the mixture was refluxed for one hour. The resultant liquid was left to cool. Then, the solvent was distilled off. Chloroform (18 ml), N-aminopyridinium iodide (0.49 g) and triethylamine (0.79 ml) were added to the resultant residue, and the mixture was stirred at room temperature for 17 hours. The resultant was put in an appropriate amount of water, and then subjected to extraction with chloroform. An organic layer was dried with anhydrous magnesium sulfate, and then the solvent was distilled off, followed by washing the resultant solid with diethyl ether to obtain the desired compound (0.5 g).

The 4-(3,3,3-trifluoro-1-propin-1-yl)benzoic acid was prepared by the method described in WO2006093832.

### Example 6

### Synthesis of 1-(4-((1E)-3,3,3-trifluoro-1-propen-1-yl)benzamide)pyridinium intramolecular salt

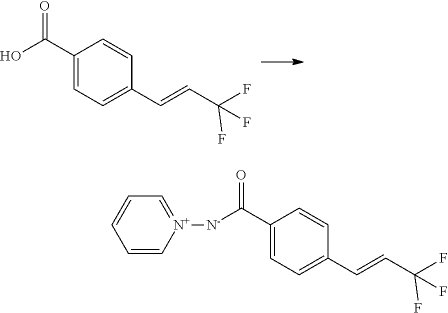

Thionyl chloride (2 ml) and pyridine (one drop) were added to 4-((1E)-3,3,3-trifluoro-1-propen-1-yl)benzoic acid (0.21 g), and then the mixture was refluxed for one hour. The resultant liquid was left to cool. The solvent was distilled off, and then chloroform (10 ml), N-aminopyridinium iodide (0.28 g) and triethylamine (0.41 ml) were added to the residue, and then the mixture was stirred at room temperature for 17 hours. The resultant was put in an appropriate amount of water, and then subjected to extraction with chloroform. An organic layer was dried with anhydrous magnesium sulfate, and then the solvent was distilled off. The resultant solid was washed with diethyl ether to obtain the desired compound (0.19 g).

The 4-((1E)-3,3,3-trifluoro-1-propen-1-yl)benzoic acid was prepared by the method described in Synthesis 1981, 5, 365.

### Example 7

### Synthesis of 1-(4-(2,2,3,3,4,4,5,5,5-nonafluoropentoxy)benzamide)pyridinium intramolecular salt

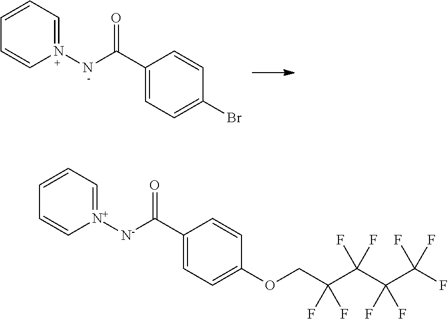

2,2,3,3,4,4,5,5,5-Nonafluoropentanol (0.28 g), tris(dibenzylideneacetone) dipalladium (33 mg), 2-di-tert-butylphosphino-2′,4′,6′-triisopropylbiphenyl (20 mg), cesium carbonate (0.24 g), and toluene (3 ml) were added to 1-(4-bromobenzamide)pyridinium intramolecular salt (0.1 g), and then reacted at 150° C. for 60 minutes using a microwave synthesizer. Toluene was distilled off from the resultant, and then the resultant was purified by silica gel column chromatography to obtain the desired compound (0.15 g).

The 1-(4-bromobenzamide)pyridinium intramolecular salt was prepared by the method described in GB1419377.

### Example 8

### Synthesis of 1-(4-((2,2,3,3,4,4,5,5,6,6,6-undecafluorohexyl)thio)benzamide) pyridinium intramolecular salt

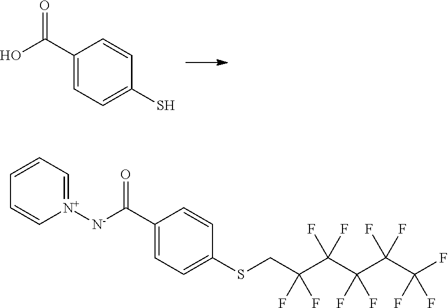 [SMILES: O=C(O)c1ccc(S)cc1 ; O=C([N-][n+]1ccccc1)c1ccc(SCC(F)(F)C(F)(F)C(F)(F)C(F)(F)C(F)(F)F)cc1]

2,2,3,3,4,4,5,5,6,6,6-Undecafluorohexyl trifulate (0.75 g), potassium carbonate (0.36 g), and DMF (6 ml) were added to 4-mercaptobenzoic acid (0.26 g), and then reacted at room temperature for two hours. Water was added to the resultant, and the mixture was subjected to extraction by ethyl acetate. An organic layer was washed with saturated saline, dried with anhydrous magnesium sulfate, filtered, and then concentrated under reduced pressure. The resultant concentrate was purified by silica gel column chromatography to obtain 4-((2,2,3,3,4,4,5,5,6,6,6-undecafluorohexyl)thio)benzoic acid (0.4 g). Thionyl chloride (1 ml) and toluene (1 ml) were added to the resultant, and then the mixture was refluxed for one hour. The resultant liquid was left to cool. Then, the solvent was distilled off. Dichloromethane (2 ml), N-aminopyridinium iodide (0.11 g) and triethylamine (0.14 ml) were added to the resultant residue, and the mixture was stirred at room temperature for 17 hours. The resultant was put in an appropriate amount of hydrochloric acid, and then subjected to extraction with ethyl acetate. An organic layer was dried with anhydrous magnesium sulfate, and then the solvent was distilled off, followed by washing the resultant solid with diethyl ether to obtain the desired compound (0.15 g).

The 2,2,3,3,4,4,5,5,6,6,6-undecafluorohexyl triflate was prepared by the method described in EP 646575.

### Example 9

### Synthesis of 1-(4-((2,2,3,3,4,4,5,5,6,6,6-undecafluorohexyl)sulfinyl)benzamide) pyridinium intramolecular salt

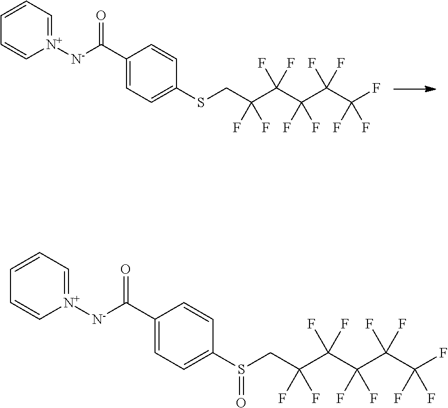 [SMILES: O=C([N-][n+]1ccccc1)c1ccc(SCC(F)(F)C(F)(F)C(F)(F)C(F)(F)C(F)(F)F)cc1 ; O=C([N-][n+]1ccccc1)c1ccc(S(=O)CC(F)(F)C(F)(F)C(F)(F)C(F)(F)C(F)(F)F)cc1]

Dichloromethane (1 ml) was added to 1-(4-((2,2,3,3,4,4,5,5,6,6,6-undecafluorohexyl)thio)benzamide)pyridinium intramolecular salt (0.12 g), and then cooled to 0° C. Meta-chloroperbenzoic acid (0.09 g) was added to the resultant, and then reacted at room temperature for five hours. An aqueous sodium bicarbonate solution was added to the resultant liquid, and then the mixture was subjected to extraction by dichloromethane. An organic layer was washed with sodium sulfite, dried with anhydrous magnesium sulfate, filtered, and then concentrated under reduced pressure. The resultant solid was washed with diethyl ether to obtain the desired compound (0.12 g).

### Example 10

### Synthesis of 1-(4-(1,1,2-trifluoro-2-(perfluoropropoxy)ethoxy)benzamide) pyridinium intramolecular salt

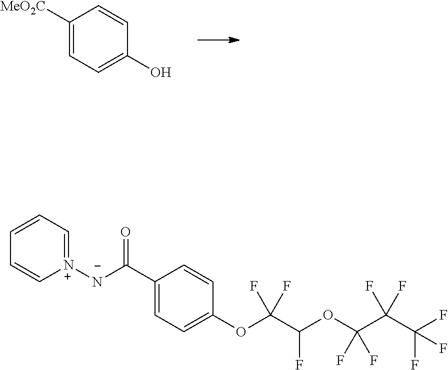 [SMILES: COC(=O)c1ccc(O)cc1 ; O=C([N-][n+]1ccccc1)c1ccc(OC(F)(F)C(F)OC(F)(F)C(F)(F)C(F)(F)F)cc1]

DMF (10 ml), potassium tert-butoxide (0.075 g), and perfluoro(propyl vinyl ether) (0.62 ml) were added to methyl 4-hydroxybenzoate (0.5 g), and reacted at room temperature for 18 hours. Water was added to the resultant liquid, and then the mixture was subjected to extraction by ethyl acetate. An organic layer was washed with saturated saline, dried with an anhydrous magnesium sulfate, filtered, and then concentrated under reduced pressure. The resultant concentrate was purified by silica gel column chromatography to obtain methyl 4-(1,1,2-trifluoro-2-(perfluoropropoxy)ethoxy)benzoate (1.2 g). THF (2 ml), ethanol (2 ml), water (8 ml), and lithium hydroxide monohydrate (0.46 g) were added thereto, and then reacted at 50° C. for 60 minutes. Hydrochloric acid was added to the resultant, and the mixture was subjected to extraction by ethyl acetate. An organic layer was washed with saturated saline, dried with anhydrous magnesium sulfate, filtered, and then concentrated under reduced pressure to obtain 4-(1,1,2-trifluoro-2-(perfluoropropoxy)ethoxy)benzoic acid (1.0 g). Thionyl chloride (1 ml), toluene (1 ml) and DMF (one drop) were added thereto, and then the mixture was refluxed for one hour. The resultant liquid was left to cool. The solvent was distilled off, and then dichloromethane (10 ml), N-aminopyridinium iodide (0.26 g) and triethylamine (0.3 ml) were added to the residue, and the mixture was stirred at room temperature for 17 hours. The resultant was put in an appropriate amount of hydrochloric acid, and then subjected to extraction with ethyl acetate. An organic layer was dried with anhydrous magnesium sulfate, and then the solvent was distilled off. The resultant residue was purified by silica gel column chromatography to obtain the desired compound (0.4 g).

### Example 11

### Synthesis of pyridin-1-ium-1-yl(4-((4-(trifluoromethoxy)phenyl)carbamoyl) benzoyl)amide

### (Step 1)

### Synthesis of (4-formylbenzoyl)(pyridin-1-ium-1-yl)amide

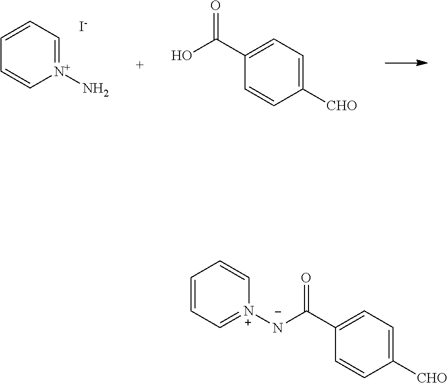 [SMILES: N[n+]1ccccc1 ; [I-] ; O=Cc1ccc(C(=O)O)cc1 ; O=Cc1ccc(C(=O)[N-][n+]2ccccc2)cc1]

4-Formylbenzoic acid (0.90 g) was dissolved in dichloromethane (20 mL), and then 1-aminopyridinium iodide (1.1 g), N-(3-dimethylaminopropyl)-N′-ethylcarbodiimide hydrochloride (1.1 g), 1-hydroxybenzotriazole hydrate (0.92 g) and triethylamine (1.2 g) were added thereto, followed by stirring the mixture at room temperature for 20 hours. Water was added to the resultant, and then the mixture was subjected to extraction by dichloromethane. The solvent was distilled off under reduced pressure to obtain the desired compound (0.84 g).

Melting point: 223° C. to 225° C.

### (Step 2)

### Synthesis of (4-carboxybenzoyl)(pyridin-1-ium-1-yl)amide

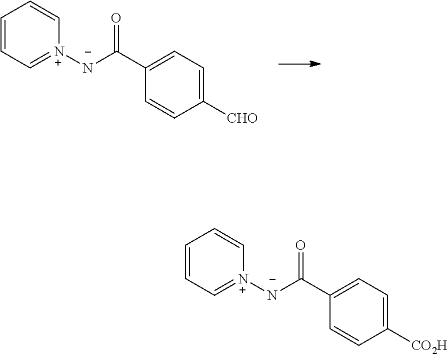 [SMILES: O=Cc1ccc(C(=O)[N-][n+]2ccccc2)cc1 ; O=C(O)c1ccc(C(=O)[N-][n+]2ccccc2)cc1]

A t-butanol (10 mL) and water (3 mL) were added to (4-formylbenzoyl)(pyridin-1-ium-1-yl)amide (0.57 g). NaH₂PO₄ (2.4 g) and isobutene (1.4 g) were added to the mixture. NaClO₂ (0.36 g) was added thereto, and stirred for five hours. The precipitate was filtered to obtain the desired compound (0.42 g).

Melting point: 230° C. to 232° C.

### (Step 3)

### Synthesis of pyridin-1-ium-1-yl(4-((4-(trifluoromethoxy)phenyl)carbamoyl) benzoyl)amide

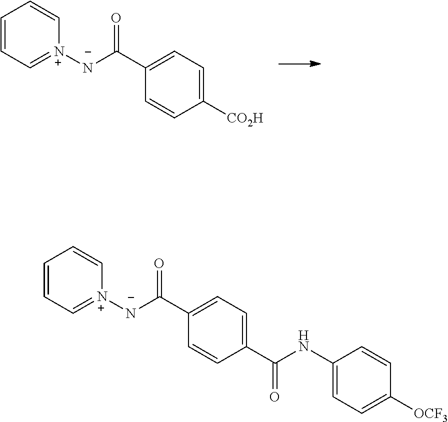 [SMILES: O=C(O)c1ccc(C(=O)[N-][n+]2ccccc2)cc1 ; O=C([N-][n+]1ccccc1)c1ccc(C(=O)Nc2ccc(OC(F)(F)F)cc2)cc1]

(4-Carboxybenzoyl)(pyridin-1-ium-1-yl)amide (0.10 g) was dissolved in dichloromethane (4 mL), and then 4-(trifluoromethoxy)aniline (0.09 g), N-(3-dimethylaminopropyl)-N′-ethylcarbodiimide hydrochloride (0.10 g), 1-hydroxybenzotriazole hydrate (0.08 g), and triethylamine (0.05 g) were added thereto, followed by stirring the mixture at room temperature for 41 hours. The precipitate was washed with water to obtain the desired compound (0.05 g).

### Example 12

### Synthesis of (E)-pyridin-1-ium-1-yl(4-(((2,2,2-trifluoroethoxy)imino)methyl) benzoyl)amide

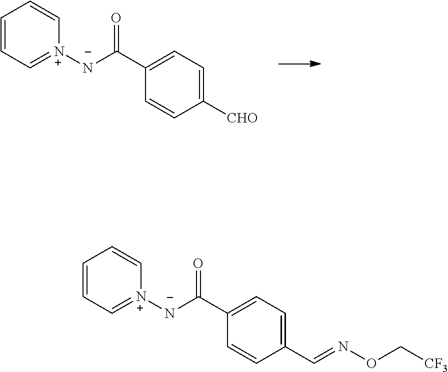 [SMILES: O=Cc1ccc(C(=O)[N-][n+]2ccccc2)cc1 ; O=C([N-][n+]1ccccc1)c1ccc(/C=N/OCC(F)(F)F)cc1]

(4-Formylbenzoyl)(pyridin-1-ium-1-yl)amide (45 mg) was dissolved in pyridine (1 mL). O-(2,2,2-trifluoroethyl)hydroxylamine (45 mg) was added thereto, and then stirred at room temperature for 24 hours. Then, the solvent was distilled off under reduced pressure. The resultant residue was purified by silica gel column chromatography to obtain the desired compound (48 mg).

### Example 13

### Synthesis of pyridin-1-ium-1-yl(4-(4-(trifluoromethoxy)phenoxy)benzoyl)amide

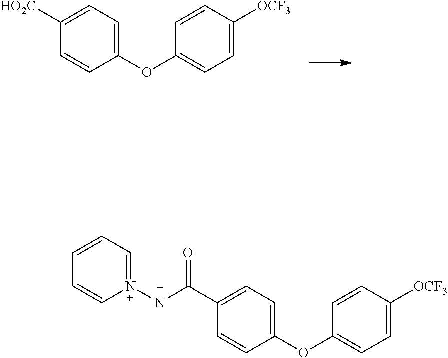 [SMILES: O=C(O)c1ccc(Oc2ccc(OC(F)(F)F)cc2)cc1 ; O=C([N-][n+]1ccccc1)c1ccc(Oc2ccc(OC(F)(F)F)cc2)cc1]

4-(4-(Trifluoromethoxy)phenoxy)benzoic acid (0.33 g) was dissolved in dichloromethane (5 mL), and then oxalyl chloride (0.15 g) and DMF (several drops) were added thereto, followed by stirring the mixture for 1.5 hours. The solvent was distilled off from the resultant under reduced pressure. The resultant was dissolved in dichloromethane (5 mL). 1-Aminopyridinium iodide (0.29 g) and triethylamine (0.13 g) were added thereto, followed by stirring the mixture at room temperature for 48 hours. Water was added to the resultant, and the mixture was subjected to extraction by ethyl acetate. An organic layer was concentrated under reduced pressure. The resultant concentrate was purified by silica gel column chromatography to obtain the desired compound (40 mg).

### Example 14

### Synthesis of pyridin-1-ium-1-yl(4-(4-(trifluoromethyl)benzyl)benzoyl)amide

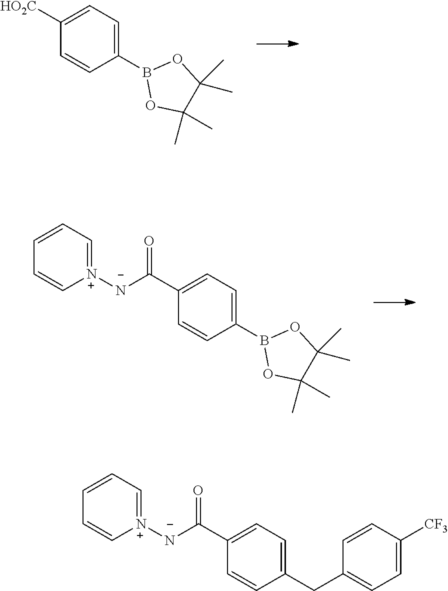 [SMILES: CC1(C)OB(c2ccc(C(=O)O)cc2)OC1(C)C ; CC1(C)OB(c2ccc(C(=O)[N-][n+]3ccccc3)cc2)OC1(C)C ; O=C([N-][n+]1ccccc1)c1ccc(Cc2ccc(C(F)(F)F)cc2)cc1]

4-(4,4,5,5-Tetramethyl-1,3,2-dioxaboran-2-yl)benzoic acid (12.4 g) was dissolved in dichloromethane (100 mL), and then oxalyl chloride (7.0 g) and DMF (several drops) were added thereto, followed by stirring the mixture for 1.5 hours. The solvent was distilled off from the resultant under reduced pressure. The resultant was dissolved in dichloromethane (150 mL). 1-Aminopyridinium iodide (12.2 g) and triethylamine (11.1 g) were added thereto, followed by stirring the mixture at room temperature for 16 hours. Then, an aqueous saturated sodium bicarbonate solution was added thereto. The precipitate was collected by filtration to obtain pyridin-1-ium-1-yl(4-(4,4,5,5-tetramethyl-1,3,2-dioxaboran-2-yl)benzoyl)amide (0.43 g).

The resultant pyridin-1-ium-1-yl(4-(4,4,5,5-tetramethyl-1,3,2-dioxaboran-2-yl)benzoyl)amide (0.26 g) was dissolved in N,N-dimethylformamide (3 mL) and water (1 mL). 1-(Bromomethyl)-4-(trifluoromethyl)benzene (0.23 g), potassium carbonate (0.17 g), and tetrakis(triphenylphosphine)palladium (92 mg) were added thereto, and then the mixture was stirred at 100° C. for 7.5 hours. Water was added thereto, and the mixture was subjected to extraction by ethyl acetate. An organic layer was concentrated under reduced pressure. The resultant concentrate was purified by silica gel column chromatography to obtain the desired compound (0.12 g).

### Example 15

### Synthesis of pyridin-1-ium-1-yl(4-((4-(trifluoromethoxy)phenoxy)methyl) benzoyl)amide

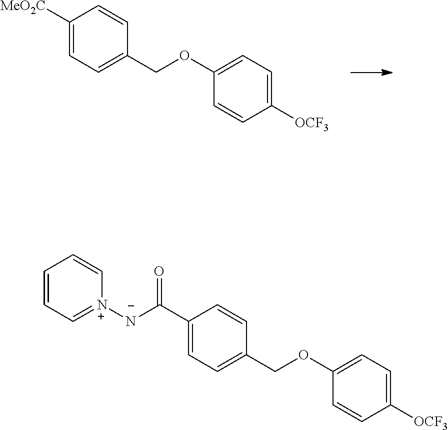 [SMILES: COC(=O)c1ccc(COc2ccc(OC(F)(F)F)cc2)cc1 ; O=C([N-][n+]1ccccc1)c1ccc(COc2ccc(OC(F)(F)F)cc2)cc1]

Methyl 4-((4-(trifluoromethoxy)phenoxy)methyl)benzoate (1.6 g) was dissolved in ethanol (15 mL), and then an aqueous solution of 4N sodium hydroxide (5 mL) was added thereto, followed by stirring the mixture at 60° C. for two hours. The resultant was neutralized with an aqueous solution of 4N hydrogen chloride. The precipitate was collected by filtration to obtain 4-((4-(trifluoromethoxy)phenoxy)methyl)benzoic acid (1.4 g).

The resultant 4-((4-(trifluoromethoxy)phenoxy)methyl)benzoic acid (0.31 g) was dissolved in dichloromethane (3 mL), and then oxalyl chloride (0.15 g) and DMF (one drop) were added thereto, followed by stirring the mixture for one hour. The solvent was distilled off from the resultant under reduced pressure, and the resultant was dissolved in dichloromethane (3 mL). 1-Aminopyridinium iodide (0.24 g) and triethylamine (0.24 g) were added thereto, followed by stirring the mixture at room temperature for 16 hours. Water was added thereto, and the mixture was subjected to extraction by ethyl acetate. An organic layer was concentrated under reduced pressure to obtain the desired compound (0.43 g).

### Example 16

### Synthesis of (4-((tert-butoxycarbonyl)amino)benzoyl)(pyridin-1-ium-1-yl)amide

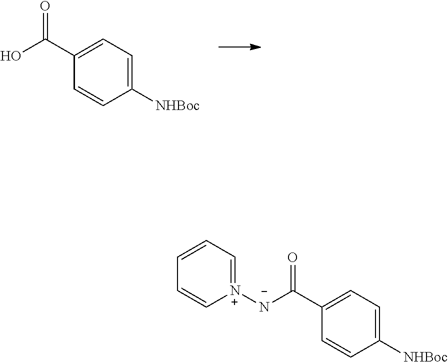 [SMILES: CC(C)(C)OC(=O)Nc1ccc(C(=O)O)cc1 ; CC(C)(C)OC(=O)Nc1ccc(C(=O)[N-][n+]2ccccc2)cc1]

4-((Tert-butoxycarbonyl)amino)benzoic acid (5.0 g) was dissolved in dichloromethane (60 mL), and then 1-aminopyridinium iodide (5.1 g), N-(3-dimethylaminopropyl)-N′-ethylcarbodiimide hydrochloride (4.8 g), 1-hydroxybenzotriazole hydrate (3.9 g), and triethylamine (5.1 g) were added thereto, followed by stirring the mixture at room temperature for 17 hours. Water was added thereto, and then the mixture was filtered. The resultant solid was washed with ethyl acetate to obtain the desired compound (6.2 g).

### Example 17

### Synthesis of 1-(4-aminobenzamide)pyridin-1-ium hydrochloride

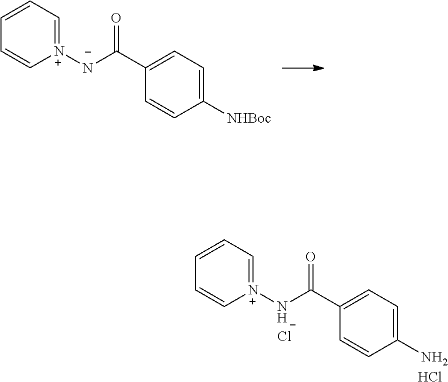 [SMILES: CC(C)(C)OC(=O)Nc1ccc(C(=O)[N-][n+]2ccccc2)cc1 ; Nc1ccc(C(=O)N[n+]2ccccc2)cc1 ; [Cl-] ; Cl]

(4-((Tert-butoxycarbonyl)amino)benzoyl)(pyridin-1-ium-1-yl)amide (3.1 g) was dissolved in dichloromethane (20 mL), and then trifluoroacetic acid (10 mL) was added thereto, followed by stirring the mixture at room temperature for 2.5 hours. The solvent was distilled off from the resultant under reduced pressure. The resultant residue was dissolved in methanol (10 mL), and then a 4N hydrochloric acid solution in dioxane (10 mL) was added thereto. The solvent was distilled off from the resultant under reduced pressure to obtain the desired compound (3.0 g).

### Example 18

### Synthesis of (4-((tert-butoxycarbonyl)(4-(trifluoromethoxy)benzyl)amino) benzoyl)(pyridin-1-ium-1-yl)amide

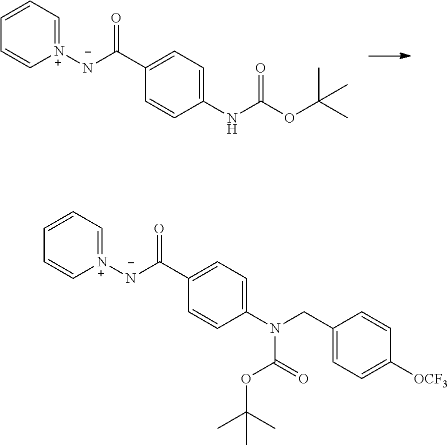 [SMILES: CC(C)(C)OC(=O)Nc1ccc(C(=O)[N-][n+]2ccccc2)cc1 ; CC(C)(C)OC(=O)N(Cc1ccc(OC(F)(F)F)cc1)c1ccc(C(=O)[N-][n+]2ccccc2)cc1]

DMF (6 mL) was added to (4-((tert-butoxycarbonyl)amino)benzoyl)(pyridin-1-ium-1-yl)amide (0.31 g), and then sodium hydride (53 mg) was added thereto at 0° C., followed by stirring the mixture for 30 minutes. 1-(Bromomethyl)-4-(trifluoromethoxy)benzene (0.28 g) was added thereto, and stirred for one hour. Water was added thereto. The precipitate was collected by filtration to obtain the desired compound (0.49 g).

### Example 19

### Synthesis of 1-(4-((4-(trifluoromethoxy)benzyl)amino)benzamido)pyridin-1-ium hydrochloride

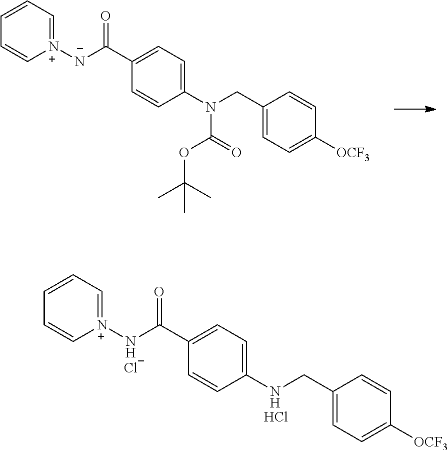 [SMILES: CC(C)(C)OC(=O)N(Cc1ccc(OC(F)(F)F)cc1)c1ccc(C(=O)[N-][n+]2ccccc2)cc1 ; O=C(N[n+]1ccccc1)c1ccc(NCc2ccc(OC(F)(F)F)cc2)cc1 ; [Cl-] ; Cl]

(4-((Tert-butoxycarbonyl)(4-(trifluoromethoxy)benzyl)amino)benzoyl)(pyridin-1-ium-1yl)amide (0.37 g) was dissolved in dichloromethane (3 mL), and then trifluoroacetic acid (1 mL) was added thereto, followed by stirring the mixture at room temperature for 18 hours. The solvent was distilled off from the resultant under reduced pressure. The resultant residue was dissolved in methanol (3 mL), and then a 4N hydrochloric acid solution in dioxane (1 mL) was added thereto. The solvent was distilled off from the resultant under reduced pressure to obtain the desired compound (0.36 g).

### Example 20

### Synthesis of pyridin-1-ium-1-yl(4-(4-(trifluoromethyl)benzamido)benzoyl)amide

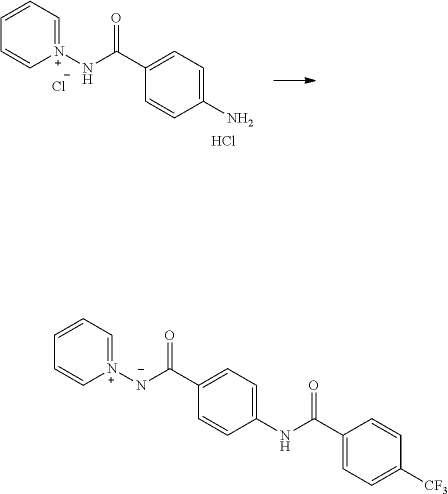 [SMILES: Nc1ccc(C(=O)N[n+]2ccccc2)cc1 ; [Cl-] ; Cl ; O=C([N-][n+]1ccccc1)c1ccc(NC(=O)c2ccc(C(F)(F)F)cc2)cc1]

1-(4-Aminobenzamide)pyridin-1-ium hydrochloride (0.20 g) was dissolved in dichloromethane (3 mL), and then 4-(trifluoromethyl)benzoyl chloride (0.18 g) and triethylamine (0.25 g) were added thereto, followed by stirring the mixture at room temperature for 16.5 hours. Water was added thereto. The precipitate was collected by filtration. The resultant precipitate was washed with ethyl acetate to obtain the desired compound (0.23 g).

### Example 21

### Synthesis of pyridin-1-ium-1-yl(4-((4-(trifluoromethyl)phenyl)sulfonamido) benzoyl)amide

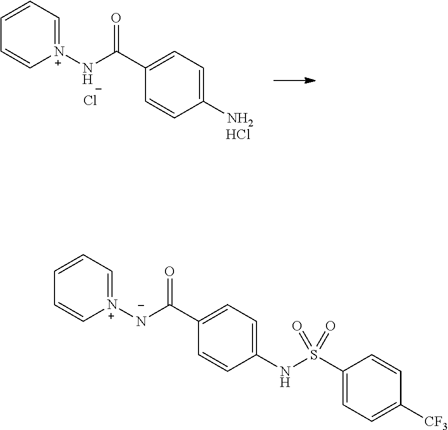 [SMILES: Nc1ccc(C(=O)N[n+]2ccccc2)cc1 ; [Cl-] ; Cl ; O=C([N-][n+]1ccccc1)c1ccc(NS(=O)(=O)c2ccc(C(F)(F)F)cc2)cc1]

1-(4-Aminobenzamide)pyridin-1-ium hydrochloride (0.20 g) was dissolved in dichloromethane (3 mL), and then 4-(trifluoromethyl)benzene sulfonyl chloride (0.21 g) and triethylamine (0.25 g) were added thereto, followed by stirring the mixture at room temperature for 16.5 hours. Water was added thereto. The precipitate was collected by filtration. The precipitate was washed with ethyl acetate to obtain the desired compound (0.15 g).

Some of the compounds according to the present invention produced in the same way as mentioned in the examples shown above are shown in Table 1. Among the compounds according to the present invention, compounds of formula (1) are indicated in Table 1. In formula (1), the number on the pyridine ring indicates the substitution position of X¹, and the number on the benzene ring indicates the substitution position of X³. In Table 1, Me indicates a methyl group, Ph indicates a phenyl group, and Py indicates a pyridyl group. The physical property of each compound is shown in the column “physical property”. The property, refractive index (nD) or melting point (m.p.) is indicated as the physical property.

The NMR data of Compound C1-68 is shown below.

¹H-NMR (CDCl₃, δ ppm) 7.01 (m, 1H), 7.27 (t, 1H), 7.31 (t, 1H), 7.42 (t, 1H), 7.65 (t, 2H), 7.86 (t, 1H), 8.92 (t, 2H).

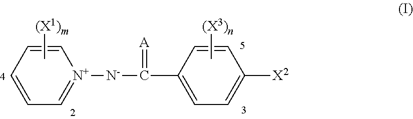

TABLE 1

| Compound | | | | | Physical |
|---|---|---|---|---|---|
| No. | (X¹)m | A | X² | (X³)n | property |
| C1-1 | — | O | CONH—(4-CF₃O—Ph) | — | m.p. 281-283° C. |
| C1-2 | — | O | CONH—CH₂CH₂CH₂CH₂SCF₃ | — | m.p. 180-190° C. |
| C1-3 | — | O | CONH—CH₂CH₂CH₂CF₃ | — | m.p. 158-160° C. |
| C1-4 | — | O | CH═N—OCH₂CF₂CF₃ | — | m.p. 139-141° C. |
| C1-5 | — | O | CH═N—OCH₂CF₃ | — | m.p. 142-143° C. |
| C1-6 | — | O | CH₂—(4-CF₃O—Ph) | — | m.p. 149-151° C. |
| C1-7 | — | O | CH₂—(4-CF₃—Ph) | — | m.p. 180-183° C. |
| C1-8 | — | O | O—(4-CF₃O—Ph) | — | m.p. 111-113° C. |
| C1-9 | — | O | CH₂—O—(4-CF₃O—Ph) | — | m.p. 174-177° C. |
| C1-10 | — | O | O—CF₂CFH—O—CF₂CF₂CF₂OCF₃ | — | m.p. 101-103° C. |
| C1-11 | — | O | O—CF₂CFH—O—CF₂CF₂CF₃ | — | m.p. 140-142° C. |
| C1-12 | — | O | O—CH₂CF₂CF₂CF₂CF₂CF₂CF₂H | — | m.p. 118-120° C. |
| C1-13 | — | O | O—CH₂CF₂CF₂CF₂CF₂CF₂H | — | m.p. 132-134° C. |
| C1-14 | — | O | O—CH₂CF₂CF₂CF₂CF₂CF₂CF₂CF₃ | — | m.p. 158-160° C. |
| C1-15 | — | O | O—CH₂CF₂CF₂CF₂CF₂CF₂CF₃ | — | m.p. 151-153° C. |
| C1-16 | — | O | O—CH₂CF₂CF₂CF₂CF₂CF₃ | — | m.p. 149-151° C. |
| C1-17 | — | O | S(═O)—CH₂CF₂CF₂CF₂CF₂CF₃ | — | m.p. 128-130° C. |
| C1-18 | — | O | S—CH₂CF₂CF₂CF₂CF₂CF₃ | — | m.p. 132-134° C. |
| C1-19 | — | O | O—CH₂CF₂CF₂CF₂CF₃ | — | m.p. 87-89° C. |
| C1-20 | — | O | O—CH₂CF₂CF₂CF₃ | — | m.p. 158-160° C. |
| C1-21 | — | O | O—CH₂CF₂CF₃ | — | m.p. 180-182° C. |
| C1-22 | — | O | O—CH₂CF₃ | — | m.p. 140-142° C. |
| C1-23 | — | O | C═C—(4-CF₃O—Ph) | — | m.p. 197-199° C. |
| C1-24 | — | O | O(5-CF₃—Py-2-yl) | — | m.p. 174-176° C. |
| C1-25 | — | O | CH═CH—CF₃ | — | m.p. 215-220° C. |
| C1-26 | — | O | C═C—CF₃ | — | m.p. 182-186° C. |
| C1-27 | — | O | CH₂—O(4-CF₃—Ph) | — | m.p. 194-197° C. |
| C1-28 | — | O | SCF₃ | — | m.p. 162-162.5° C. |
| C1-29 | — | O | C═C—(4-CF₃—Ph) | — | m.p. 223-226° C. |
| C1-30 | — | O | CO(4-CF₃O—Ph) | — | m.p. 125-128° C. |
| C1-31 | — | O | CH═CH—(4-CF₃—Ph) | — | m.p. 250° C. |
| C1-32 | — | O | O—CH₂(4-CF₃—Ph) | — | m.p. 214-216° C. |
| C1-33 | — | O | O—CH₂(4-Br—Ph) | — | m.p. 211-214° C. |
| C1-34 | — | O | O—CH₂(4-CF₃O—Ph) | — | m.p. 189-191° C. |
| C1-35 | — | O | CONHPh | — | m.p. 250° C. |
| C1-36 | — | O | CONMe₂ | — | m.p. 199-201° C. |
| C1-37 | — | O | SO₂Me | — | m.p. 228-230° C. |
| C1-38 | — | O | O—CH₂—CH═CCl(Me) | — | m.p. 151-153° C. |
| C1-39 | — | O | O—CH₂—C(Cl)═CHCl | — | m.p. 95-98° C. |
| C1-40 | — | O | O—CH₂—C(Me)═CH₂ | — | m.p. 105-108° C. |
| C1-41 | — | O | O—CH₂—CH═C(Me)₂ | — | m.p. 120-122° C. |
| C1-42 | — | O | O—CH₂—CCH | — | m.p. 103-106° C. |
| C1-43 | — | O | O—CH₂—CH═CCl₂ | — | m.p. 95-98° C. |
| C1-44 | — | O | O—CH₂—CH═CH₂ | — | m.p. 120-122° C. |
| C1-45 | — | O | OCH₂Ph | — | m.p. 148-152° C. |
| C1-46 | — | O | CO₂Me | — | m.p. 186-187° C. |
| C1-47 | — | O | O—(4-CF₃—Ph) | — | m.p. 140-142° C. |
| C1-48 | — | O | O—(3-Cl—5-CF₃—Py-2-yl) | — | m.p. 170-172° C. |
| C1-49 | — | O | OCF₃ | — | m.p. 141-143° C. |
| C1-50 | — | O | NH—CONH—(2,4-Cl₂—Ph) | — | m.p. 264-266° C. |
| C1-51 | — | O | NH—CSNH—(4-CF₃—Ph) | — | m.p. 204-206° C. |
| C1-52 | — | O | N(Me)—CO—(4-CF₃—Ph) | — | m.p. 208-209° C. |
| C1-53 | — | O | CH═N—NH—SO₂—(4-Me—Ph) | — | m.p. 202-204° C. |
| C1-54 | — | O | NH—SO₂—(4-CF₃—Ph) | — | m.p. 199-203° C. |
| C1-55 | — | O | NH—CO—(4-CF₃—Ph) | — | m.p. 287-289° C. |
| C1-57 | — | O | NH—CO₂^{t}Bu | — | m.p. 251-253° C. |
| C1-58 | — | O | CH₂CH₂CF₂CF₂CF₂CF₃ | 3-F | m.p. 135-137° C. |
| C1-59 | — | O | CH═CHCF₂CF₂CF₂CF₃ | 3-F | m.p. 160-165° C. |
| C1-60 | — | O | C(Me)═N—OCH₂CF₂CF₂CF₂CF₃ | — | m.p. 143-145° C. |
| C1-61 | — | O | C(Me)═N—OCH2CF2CF2CF3 | — | m.p. 133-135° C. |
| C1-62 | — | O | CH═N—OCH₂CF₂CFHCF₃ | 3-F | m.p. 87-89° C. |
| C1-63 | — | O | CH═N—OCH₂CF₂CF₂CF₂CF₃ | 3-F | m.p. 96-98° C. |
| C1-64 | — | O | CH═N—OCH₂CF₂CF₂CF₃ | 3-F | m.p. 110-112° C. |
| C1-65 | — | O | CH═N—OCH₂CF₂CF₃ | 3-F | m.p. 119-121° C. |
| C1-66 | — | O | O—CH₂CH₂CH₂CH₂CH₂CH₂SCF₃ | 3-F | m.p. 91-93° C. |
| C1-67 | — | O | O—CH₂CH₂CH₂CH₂CH₂CF₃ | 3-F | m.p. 122-123° C. |
| C1-68 | — | O | O—CF₂CFH—O—CF₂CF(CF₃)OCF₂CF₂CF₃ | 3-F | — |
| C1-69 | — | O | O—CH₂CF₂CF₂CF₂CF₂CF₂CF₂H | 3,5-F₂ | m.p. 151-153° C. |
| C1-70 | — | O | O—CH₂CF₂CF₂CF₂CF₂CF₂CF₃ | 3,5-F₂ | m.p. 166-168° C. |
| C1-71 | — | O | O—CH₂CF₂CF₂CF₂CF₃ | 3-Cl | m.p. 166-168° C. |
| C1-72 | — | O | O—CH₂CF₂CF₂CF₂CF₂CF₂CF₂H | 3-Cl | m.p. 155-157° C. |
| C1-73 | — | O | O—CH₂CF₂CF₂CF₂CF₂CF₂CF₃ | 3-Cl | m.p. 176-178° C. |
| C1-74 | — | O | O—CF₂CFH—O—CF₂CF₂CF₂OCF₃ | 3-F | nD(22.1)1.6295 |
| C1-75 | — | O | O—CH₂CF₂CF₂CF₂CF₂CF₂CF₂H | 3-F | m.p. 161-163° C. |
| C1-76 | — | O | O—CH₂CF₂CF₂CF₂CF₂CF₂CF₃ | 3-F | m.p. 172-174° C. |
| C1-77 | — | O | C═C—(4-CF₃O—Ph) | 3-Cl | m.p. 154-156° C. |
| C1-78 | — | O | C═C—(4-CF₃O—Ph) | 3-F | m.p. 140-142° C. |

Additional examples of the compound according to the present invention prepared in the same way as mentioned above are shown in Table 2.

TABLE 2

| Compound | | Physical |
|---|---|---|
| No. | Formula | property |
| C2-1 | 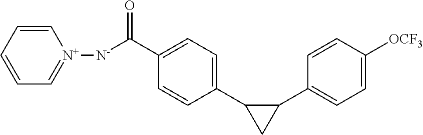 [SMILES: O=C([N-][n+]1ccccc1)c1ccc(C2CC2c2ccc(OC(F)(F)F)cc2)cc1] | m.p. 93-95° C. |
| C2-2 | 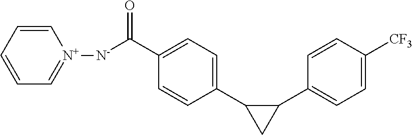 [SMILES: O=C([N-][n+]1ccccc1)c1ccc(C2CC2c2ccc(C(F)(F)F)cc2)cc1] | m.p. 162-164° C. |
| C2-3 | 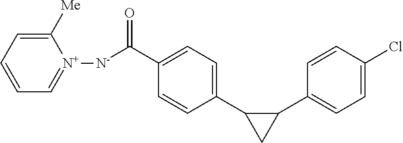 [SMILES: Cc1cccc[n+]1[N-]C(=O)c1ccc(C2CC2c2ccc(Cl)cc2)cc1] | m.p. 140-144° C. |
| C2-4 | 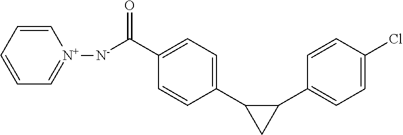 [SMILES: O=C([N-][n+]1ccccc1)c1ccc(C2CC2c2ccc(Cl)cc2)cc1] | m.p. 161-165° C. |
| C2-5 | 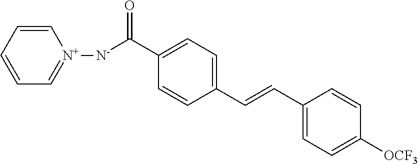 [SMILES: O=C([N-][n+]1ccccc1)c1ccc(/C=C/c2ccc(OC(F)(F)F)cc2)cc1] | m.p. 241-243° C. |
| C2-6 | 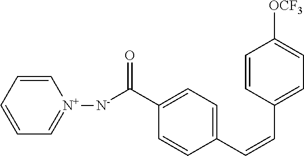 [SMILES: O=C([N-][n+]1ccccc1)c1ccc(/C=C\c2ccc(OC(F)(F)F)cc2)cc1] | m.p. 123-125° C. |
| C2-7 | 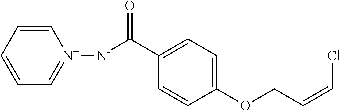 [SMILES: O=C([N-][n+]1ccccc1)c1ccc(OC/C=C\Cl)cc1] | m.p. 101-104° C. |
| C2-8 | 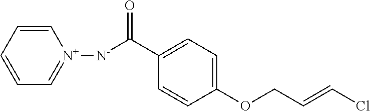 [SMILES: O=C([N-][n+]1ccccc1)c1ccc(OC/C=C/Cl)cc1] | m.p. 161-162° C. |
| C2-9 | 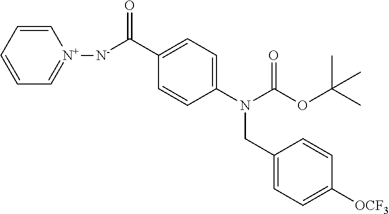 [SMILES: CC(C)(C)OC(=O)N(Cc1ccc(OC(F)(F)F)cc1)c1ccc(C(=O)[N-][n+]2ccccc2)cc1] | m.p. 183-184° C. |
| C1-56 | 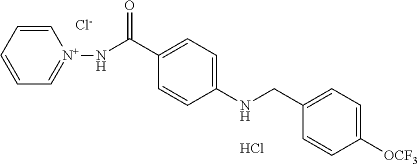 [SMILES: O=C(N[n+]1ccccc1)c1ccc(NCc2ccc(OC(F)(F)F)cc2)cc1 ; [Cl-] ; Cl] | m.p. 204-206° C. |

### (Biological Test)

The following test examples show that the compound according to the present invention is useful as an active ingredient of a pest control agent. The term “part” is based on the mass.

### (Preparation of Test Emulsion)

5 parts by mass of a compound according to the present invention, 93.6 parts of dimethylformamide, and 1.4 parts of polyoxyethylene alkyl aryl ether were mixed and dissolved to obtain an emulsion (1) containing 5% by mass of the active ingredient.

(1) Efficacy Test (1) Against Mythimna separata Using Artificial Feed

0.8 g of commercially-available artificial feed (Insect LFS, manufactured by Nosan Corporation) and 1 μl of the emulsion (I) were mixed well, and then 0.2 g of the mixture per treated area was packed into plastic test containers (each having a capacity of 1.4 ml) as test feeds.

Two second-instar larvae of Mythimna separata were left per treated area, and then the test containers were sealed with plastic covers. The test containers were placed in a thermostatic chamber at 25° C., and, after five days had passed therefrom, the mortality and the feed intake were measured. The test was repeated twice. In addition, the test was conducted under the same conditions as described above, except that the compound according to the present invention was removed from the emulsion (I), and evaluated as a solvent control area.

TABLE 3
Compound No.

| | | | |
|---|---|---|---|
| C1-4 | C1-20 | C2-2 | C1-72 |
| C1-9 | C1-21 | C2-4 | C1-73 |
| C1-10 | Cl-23 | C2-5 | Cl-74 |
| C1-11 | C1-25 | C1-62 | C1-75 |
| C1-12 | C1-27 | C1-64 | C1-76 |
| C1-13 | C1-29 | C1-65 | C1-77 |
| C1-14 | C1-31 | C1-68 | C1-78 |
| C1-15 | C1-32 | C1-69 | |
| C1-16 | C1-34 | C1-70 | |
| C1-19 | C1-48 | C1-71 | |

The compounds shown in Table 3 were subjected to an efficacy test against Mythimna separata. All of the compounds caused 100% mortality of Mythimna separata or 10% or less of the feed intake, relative to the feed intake at the solvent control area, and thus the efficacy thereof against Mythimna separata was confirmed.

(2) Efficacy Test (2) Against Mythimna separata

The emulsion (1) was diluted with water such that the amount of the compound according to the present invention became 125 ppm by mass. A corn leaf disc was immersed in the diluted solution for 30 seconds. The corn leaf disc was put in a petri dish, and five second-instar larvae of Mythimna separata were left therein. The petri dish was placed in a thermostatic chamber at a temperature of 25° C. and a humidity of 60%. After six days had passed from leaving the larvae, life or death was assessed and the mortality was calculated. The test was repeated twice.

TABLE 4
Compound No.
C1-27
C1-29
C1-31
C1-32
C1-33
C1-34
C1-47
C1-49
C2-4

The compounds shown in Table 4 were subjected to an efficacy test against Mythimna separata. All of the compounds caused 80% or more mortality of Mythimna separata.

(3) Efficacy Test Against Spodoptera litura

The emulsion (1) was diluted with water such that the amount of the compound according to the present invention became 125 ppm by mass. A cabbage leaf disc was immersed in the diluted solution for 30 seconds. The cabbage leaf disc was air-dried and then put in a petri dish, followed by leaving five second-instar larvae of Spodoptera litura therein. The petri dish was placed in a thermostatic chamber at a temperature of 25° C. and a humidity of 60%. After six days had passed from leaving the larvae, life or death was assessed and the mortality was calculated. The test was repeated twice.

TABLE 5
Compound No.

| | | | |
|---|---|---|---|
| C1-6 | C1-28 | C2-5 | C1-77 |
| C1-10 | Cl-29 | Cl-58 | |
| C1-11 | C1-31 | C1-59 | |
| C1-12 | C1-32 | C1-63 | |
| C1-14 | C1-34 | C1-64 | |
| C1-15 | C1-47 | C1-69 | |
| C1-16 | C1-48 | C1-70 | |
| C1-19 | C1-49 | C1-74 | |
| C1-23 | C2-1 | C1-75 | |
| C1-25 | C2-2 | C1-76 | |

The compounds shown in Table 5 were subjected to an efficacy test against Spodoptera litura. All of the compounds caused 80% or more mortality of Spodoptera litura.

(4) Efficacy Test Against Helicoverpa armigera

The emulsion (I) was diluted with water such that the amount of the compound according to the present invention became 125 ppm by mass. A cabbage leaf disc was immersed in the diluted solution for 30 seconds. The cabbage leaf disc was air-dried and then put in a petri dish, followed by leaving five second-instar larvae of Spodoptera litura therein. The petri dish was placed in a thermostatic chamber at a temperature of 25° C. and a humidity of 60%. After six days had passed from leaving the larvae, life or death was assessed and the mortality was calculated. The test was repeated twice.

TABLE 6
Compound No.
C1-10
C1-11
C1-15
C1-23
C2-1
C2-2
C1-60
C1-61
C1-63
C1-64

The compounds shown in Table 6 were subjected to an efficacy test against Helicoverpa armigera. All of the compounds caused 80% or more mortality of Helicoverpa armigera.

(5) Efficacy Test Against Plutella xylostella

The emulsion (I) was diluted with water such that the amount of the compound according to the present invention became 125 ppm by mass. A cabbage leaf disc was immersed in the diluted solution for 30 seconds. The cabbage leaf disc was air-dried and then put in a petri dish, followed by leaving five second-instar larvae of Spodoptera litura therein. The petri dish was placed in a thermostatic chamber at a temperature of 25° C. and a humidity of 60%. After three days had passed from leaving the larvae, life or death was assessed and the mortality was calculated. The test was repeated twice.

TABLE 7
Compound No.
C1-27
C1-29
C1-30
C1-31
C1-32
C1-33
C1-49
C2-5

The compounds shown in Table 7 were subjected to an efficacy test against Plutella xylostella. All of the compounds caused 80% or more mortality of Plutella xylostella.

(6) Efficacy Test Against Tetranychus kanzawai

Five female adults of Tetranychus kanzawai were left on primary leaves of mung bean planted in a 3-sun pot. The emulsion (I) was diluted with water such that the amount of the compound according to the present invention became 125 ppm by mass. The diluted solution was sprayed on the mung bean and evaluated as a treated area. In addition, the test was conducted under the same conditions as described above, except that the compound according to the present invention was removed from the emulsion (I), and evaluated as a solvent control area. The 3-sun pot was placed in a thermostatic chamber at a temperature of 25° C. and a humidity of 65%, and after four days had passed from spraying, the female adults of Tetranychus kanzawai were removed to leave only laid eggs remaining on the leaves. After eleven days had passed from spraying, the number of living Tetranychus kanzawai was counted and the control ratio was calculated in accordance with the following formula. The test was repeated twice.

Control ratio (%)=100×[1−Nt/Nc]

Nt is the living number at the treated area, and Nc is the living number at the solvent control area.

TABLE 8
Compound No.

| | | |
|---|---|---|
| C1-4 | C2-3 | C1-77 |
| C1-6 | C1-62 | C1-78 |
| C1-7 | C1-63 | |
| C1-8 | C1-64 | |
| C1-9 | C1-65 | |
| C1-10 | C1-66 | |
| C1-11 | C1-67 | |
| C1-18 | C1-68 | |
| C1-23 | C1-73 | |
| C2-2 | C1-74 | |

The compounds shown in Table 8 were subjected to an efficacy test against Tetranychus kanzawai. All of the compounds exhibited 90% or more control ratio.

(7) Efficacy Test Against Tetranychus urticae

Five female adults of Tetranychus urticae were left on primary leaves of kidney bean planted in a 3-sun pot. The emulsion (I) was diluted with water such that the amount of the compound according to the present invention became 125 ppm by mass. The diluted solution was sprayed on the kidney bean and evaluated as a treated area. In addition, the test was conducted under the same conditions as described above, except that the compound according to the present invention was removed from the emulsion (1), and evaluated as a solvent control area. The 3-sun pot was placed in a thermostatic chamber at a temperature of 25° C. and a humidity of 65%, and after three days had passed from spraying, the female adults of Tetranychus urticae were removed to leave only laid eggs remaining on the leaves. After ten days had passed from spraying, the number of living Tetranychus urticae was counted and the control ratio was calculated in accordance with the following formula. The test was repeated twice.

Control ratio (%)=100×[1−Nt/Nc]

Nt is the living number at the treated area, and Nc is the living number at the solvent control area.

TABLE 9
Compound No.

| | |
|---|---|
| C1-24 | C2-4 |
| C1-27 | C2-5 |
| C1-28 | C2-6 |
| C1-29 | |
| C1-34 | |
| C1-37 | |
| C1-47 | |
| C1-48 | |
| C1-49 | |
| C2-3 | |

The compounds shown in Table 9 were subjected to an efficacy test against Tetranychus urticae. All of the compounds exhibited 90% or more control ratio.

(8) Efficacy Test Against Aphis gossypii

Cucumber was sown in a 3-sun pot. After ten days had passed from germination, a female adult of Aphis gossypii was left on the cucumber. On the next day, laid first-instar larvae were left, and the female adult was removed. The emulsion (I) was diluted with water such that the amount of the compound according to the present invention became 125 ppm by mass. The diluted solution was sprayed on the cucumber. Then, the cucumber was placed in a thermostatic chamber at a temperature of 25° C. and a humidity of 60%. After five days had passed therefrom, life or death of Aphis gossypii was assessed and the mortality was calculated.

Compounds C-63 and C-74 were subjected to an efficacy test against Aphis gossypii. Both of the compounds caused 80% or more mortality.

(9) Efficacy Test Against Nilaparvata lugens

The emulsion (I) was diluted with water such that the amount of the compound according to the present invention became 125 ppm by mass. A rice seedling was immersed in the diluted solution for 30 seconds, air-dried, and then placed in a plastic case, followed by leaving five second-instar larvae of Nilaparvata lugens therein. The plastic case was placed in a thermostatic chamber at a temperature of 25° C. and a humidity of 65%. After ten days had passed therefrom, life or death was assessed and the mortality was calculated using the following formula. The test was repeated twice.

Mortality (%)=(the number of dead insects/the number of supplied insects)×100

Compounds C-63, C-64 and C-65 were subjected to an efficacy test against Nilaparvata lugens. All of the compounds caused 80% or more mortality.

Since the compounds randomly selected from the pyridinium salts according to the present invention exhibited the above-described effects, it is understood that the pyridinium salt according to the present invention, involving aspects of the compound that are not shown in the above examples, is a compound that exhibits pest control effects, acaricidal effects, and particularly insecticidal effects.

### INDUSTRIAL APPLICABILITY

The pyridinium salt according to the present invention has control activity against pests that cause problems to agricultural crops or in the health field. The control agent containing the pyridinium salt according to the present invention can effectively control pests, particularly agricultural insect pests and acarians, at low doses, and can further effectively control ectoparasites and endoparasites that may harm humans and animals.

1. A compound of formula (I) or formula (II): 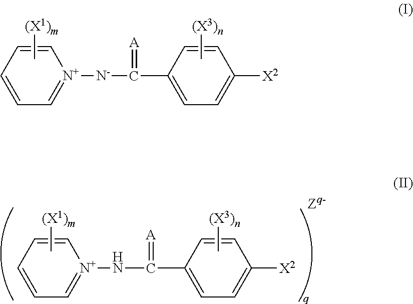
in the formulae (I) and (II),
A is an oxygen atom or a sulfur atom,
X¹ is a halogeno group, a substituted or unsubstituted C1-6 alkyl group, a substituted or unsubstituted C2-6 alkenyl group, a substituted or unsubstituted C2-6 alkynyl group, a substituted or unsubstituted C1-6 alkoxy group, a substituted or unsubstituted C2-6 alkenyloxy group, a substituted or unsubstituted C2-6 alkynyloxy group, a substituted or unsubstituted C1-6 alkylcarbonyl group, a substituted or unsubstituted C1-6 alkoxycarbonyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryl group, a substituted or unsubstituted C6-10 aryloxy group, a substituted or unsubstituted 5- or 6-membered heteroaryl group, a substituted or unsubstituted 5- or 6-membered heteroaryloxy group, a substituted or unsubstituted C6-10 arylcarbonyl group, a substituted or unsubstituted 5- or 6-membered heteroarylcarbonyl group, a group of R^{a}R^{b}N—, a group of R^{a}R^{b}N—CO—, a group of R^{c}CO—NH—, a pentafluorosulfanyl group, a nitro group, or a cyano group,
m indicates a number of X¹ and is an integer of 0 to 5,
R^{a} is a hydrogen atom, a substituted or unsubstituted C1-6 alkyl group, or a substituted or unsubstituted C6-10 aryl group,
R^{b} is a hydrogen atom, a substituted or unsubstituted C1-6 alkyl group, or a substituted or unsubstituted C6-10 aryl group,
R^{c} is a hydrogen atom, a substituted or unsubstituted C1-6 alkyl group, or a substituted or unsubstituted C6-10 aryl group,
X² is a C5-8 alkyl group, a C2-8 haloalkyl group, a (substituted or unsubstituted C6-10 aryl) C1-6 alkyl group, a (substituted or unsubstituted 5- or 6-membered heteroaryl) C1-6 alkyl group, a (substituted or unsubstituted C6-10 aryloxy) C1-6 alkyl group, a (substituted or unsubstituted 5- or 6-membered heteroaryloxy) C1-6 alkyl group, a C3-6 alkenyl group, a C2-6 haloalkenyl group, a (substituted or unsubstituted C6-10 aryl) C2-6 alkenyl group, a (substituted or unsubstituted 5- or 6-membered heteroaryl) C2-6 alkenyl group, a C2-6 alkynyl group, a C2-6 haloalkynyl group, a (substituted or unsubstituted C6-10 aryl) C2-6 alkynyl group, a (substituted or unsubstituted 5- or 6-membered heteroaryl) C2-6 alkynyl group, a hydroxy group, a substituted C1-8 alkoxy group, a substituted or unsubstituted C2-6 alkenyloxy group, a substituted or unsubstituted C2-6 alkynyloxy group, a C2-6 alkylcarbonyl group, a substituted C1-6 alkylcarbonyl group, a substituted or unsubstituted C1-6 alkoxycarbonyl group, a substituted or unsubstituted C1-6 alkylthio group, a substituted or unsubstituted C1-6 alkylsulfinyl group, a substituted or unsubstituted C1-6 alkylsulfonyl group, a substituted or unsubstituted C3-8 cycloalkyl group, a substituted or unsubstituted C6-10 aryloxy group, a substituted or unsubstituted 5- or 6-membered heteroaryloxy group, a substituted or unsubstituted C6-arylcarbonyl group, a substituted or unsubstituted 5- or 6-membered heteroarylcarbonyl group, a carbamoyl group, a group of R^{a1}R^{b1}N—, a group of R^{a1}R^{b1}N—CO—, a group of R^{a1}R^{b1}N—CO—N(R^{d1})—, a group of R^{a1}R^{b1}N—CS—N(R^{d1})—, a group of R^{c1}CO—N(R^{d1})—, a group of R^{e1}O—N═CR^{f1}—, a group of R^{g1}SO₂—N(R^{d1})—, a group of R^{g1}SO₂NH—N═CR^{f1}—, or a pentafluorosulfanyl group,
R^{a1} is a substituted or unsubstituted C1-6 alkyl group, or a substituted or unsubstituted C6-10 aryl group,
R^{b1} is a hydrogen atom, a substituted or unsubstituted C1-6 alkyl group, or a substituted or unsubstituted C6-10 aryl group,
R^{a1} and R^{b1} may be bonded together to form a divalent organic group,
R^{c1} is a hydrogen atom, a substituted or unsubstituted C1-6 alkyl group, a substituted or unsubstituted C1-6 alkoxy group, or a substituted or unsubstituted C6-10 aryl group,
R^{d1} is a hydrogen atom, a substituted or unsubstituted C1-6 alkyl group, or a substituted or unsubstituted C6-10 aryl group,
R^{e1} is a hydrogen atom, a substituted or unsubstituted C1-6 alkyl group, or a substituted or unsubstituted C6-10 aryl group,
R^{f1} is a hydrogen atom or a substituted or unsubstituted C1-6 alkyl group,
R^{g1} is a substituted or unsubstituted C1-6 alkyl group, or a substituted or unsubstituted C6-10 aryl group,
X³ is a halogeno group, a substituted or unsubstituted C1-6 alkyl group, a hydroxy group, a substituted or unsubstituted C1-6 alkoxy group, a substituted or unsubstituted C1-6 alkylthio group, a substituted or unsubstituted C1-6 alkylsulfinyl group, a substituted or unsubstituted C1-6 alkylsulfonyl group, a pentafluorosulfanyl group, a nitro group, or a cyano group,
n indicates a number of X³ and is an integer of 0 to 4,
Z^{q-} is a counter ion, and
q is a valence of the counter ion and is 1 or 2).

2. A pest control agent comprising at least one selected from compounds of claim 1 as an active ingredient thereof.

3. An insecticide or acaricide comprising at least one selected from compounds of claim 1 as an active ingredient thereof.

4. An ectoparasite control agent comprising at least one selected from compounds of claim 1 as an active ingredient thereof.

5. An endoparasite control agent or expellant comprising at least one selected from compounds of claim 1 as an active ingredient thereof.
